(19) United States
(12) Patent Application Publication
(10) Pub. No.: US 2017/0000850 A1
(43) Pub. Date: Jan. 5, 2017

(54) DIFFERENTIATION THERAPY WITH CD137 LIGAND AGONISTS

(71) Applicant: NATIONAL UNIVERSITY OF SINGAPORE, Singapore (SG)

(72) Inventors: Herbert SCHWARZ, Singapore (SG); Cheong Kin CHENG, Singapore (SG)

(21) Appl. No.: 15/106,806

(22) PCT Filed: Dec. 18, 2014
(86) PCT No.: PCT/SG2014/000606

§ 371 Date: Jun. 20, 2016

(30) Foreign Application Priority Data
Dec. 20, 2013 (SG) 201309454-5

(51) Int. Cl. A61K 38/17; C07K 16/28

(52) U.S. Cl. CPC A61K 38/1793; C07K 16/2878

(57) ABSTRACT

There is disclosed a method of treating cancer comprising administering an agonist of CD137 ligand (CD137L) to a subject in need thereof.

### TECHNICAL FIELD

The present invention generally relates to agonists of CD137 ligand (CD137L) and their use in cancer therapy. The present invention also relates to use of agonists of CD137L for inducing differentiation in cancer cells and for delivery of a therapeutic or diagnostic agent to a cell in need thereof.

### BACKGROUND

CD137 is a member of the TNF receptor family that can be expressed by a range of leukocytes, including activated T cells, NK cells and inflamed vascular endothelial cells (Thum, et al 2009). Its best characterized activity is that of T cell co-stimulation, and CD137 agonists potently enhance immune responses (Lee and Croft 2009, Tamada and Chen 2006, Wang, et al 2009).

CD137 ligand (CD137L) belongs to the TNF family, and is expressed widely and constitutively, including by antigen presenting cells (APC) as a transmembrane protein on the cell surface. APC use the CD137 receptor/ligand system to co-stimulate T cell activity.

CD137L is not only able to send a signal, i.e. by crosslinking CD137 but can also transmit signals into the cells that it is expressed on, a process referred to as “reverse signaling” (Eissner, et al 2004). “Bidirectional signaling,” i.e. signaling through CD137 as well as CD137L, occurs when CD137-expressing and CD137L-expressing cells interact (Schwarz 2005).

Reverse signaling by CD137L has been shown to induce proliferation of hematopoietic progenitor cells and their differentiation to myeloid cells, especially monocytic cells, while granulocytic differentiation is inhibited (Jiang, et al 2008a, Jiang and Schwarz 2010, Jiang, et al 2008b). CD137L signaling also affects mature myeloid cells. It activates monocytes, resulting in increased adhesion, cytokine secretion and migration, and enhances their survival and proliferation (Langstein, et al 2000, Langstein, et al 1998, Langstein, et al 1999, Langstein and Schwarz 1999). Further, CD137L signaling induces maturation of immature dendritic cells (Kim, et al 2002, Laderach, et al 2003, Lippert, et al 2008), and even differentiation of peripheral human monocytes to mature dendritic cells (DCs) (Ju, et al 2009, Kwajah and Schwarz 2010). These activities of CD137 play a role during infection-induced myelopoiesis (Tang, et al 2013) and mark CD137 as a potent monocytic differentiation factor.

Acute myeloid leukemia (AML) is characterized by a differentiation block in developing myeloid cells leading to the accumulation of immature cells that retain a high proliferation rate (Stone, et al 2004). All-trans-retinoic acid (ATRA) and arsene trioxide (ATO) are effective in inducing differentiation of acute promyelocytic leukemia (APL), one of the subtypes of AML, making them a standard therapy for APL. However, ATRA and ATO have no therapeutic effect on the remaining AML subtypes (Petrie, et al 2009). A large number of other compounds, including cytokines, have been explored for a similar differentiative effect on AML aside from APL, and many induced differentiation in monocytic cell lines, but none has been found to be of sufficient efficacy in primary AML cells (Koeffler 2010).

There is a need to provide therapeutic agents and methods for treating AML that overcome, or at least ameliorate, one or more of the disadvantages described above.

There is a need to provide a formulation of the therapeutic agents for treating AML that can be produced easily, and that is in a form that is suitable for administration to a human patient while retaining efficacy of the agent.

### SUMMARY

According to a first aspect, there is provided a method of treating cancer comprising administering an agonist of CD137 ligand (CD137L) to a subject in need thereof.

Advantageously, the agonist is capable of crosslinking CD137L to initiate reverse CD137L signaling in malignant cells, resulting in a two-fold therapeutic effect: (a) increased differentiation of malignant cells which reduces or prevents their proliferative potential thereby slowing tumour progression, and (b) inducing differentiation of the malignant cells to cells with DC-like properties which results in tumour cells with enhanced T cell-activating abilities.

Advantageously, the agonist is capable of inducing differentiation of malignant cells from all AML subtypes, including subtypes M1, M2, M5a and M5b, compared to conventional therapy for AML which only induces differentiation of AML subtype M3 cells.

According to a second aspect, there is provided an agonist of CD137 ligand (CD137L) for use in treating cancer.

According to a third aspect, there is provided the use of an agonist of CD137 ligand (CD137L) in the manufacture of a medicament for treating cancer.

According to a fourth aspect, there is provided method for inducing differentiation in cancer cells, comprising administering an agonist of CD137 ligand (CD137L) as defined herein to the cancer cells.

According to a fifth aspect, there is provided a method for delivering a therapeutic or diagnostic agent to a cell in need thereof, comprising coupling an agonist of CD137 ligand (CD137L) as defined herein to an agent selected from the group consisting of cytotoxic drugs, radiotherapeutics, chemotherapeutics, enzyme, and a carrier molecule.

### Definitions

The following words and terms used herein shall have the meaning indicated:

The terms “treat” and “treatment” refer to both therapeutic treatment and prophylactic or preventative measures, wherein the object is to prevent or slow down (lessen) an undesired physiological condition, disorder or disease, or to obtain beneficial or desired clinical results. Where the treatment relates to cancer, the term “treatment” also includes: (i) the prevention or inhibition of cancer or cancer recurrence, (ii) the reduction or elimination of symptoms or cancer cells, and (iii) the substantial or complete elimination of the cancer in question. Treatment may be effected prophylactically (prior to cancer onset) or therapeutically (following cancer diagnosis). Treatment may entail treatment with a single agent (for example, an agonist of CD137 ligand as disclosed herein) or with a combination (two or more) of agents. An “agent” is used herein broadly to refer to, for example, a compound, a molecule (such as a biomolecule) or other means for treatment, such as radiation treatment or surgery.

The term “agonist” when used with reference to a CD137L means a molecule which induces or activates the biological activity of CD137 ligand. Such agonists may include proteins, nucleic acids, carbohydrates, small molecules, antibodies or fragments thereof, aptamers, antibody mimetics, or any other compound or composition which modulates the activity of a CD137 ligand either by directly interacting with the CD137 ligand or by acting on components of the CD137 ligand signaling pathway.

The term “ligand” refers to a molecule (such as a protein) that is capable of binding to another molecule of interest. For example, a CD137 ligand may bind to the CD137 receptor and by doing so, induces signaling of the CD137 receptor. At the same time, due to bidirectional signaling, the CD137 ligand may be induced by the CD137 receptor to signal into the cell on which the CD137 ligand is expressed. Hence, in the context of the CD137 receptor/ligand system, both the CD137L and CD137 receptor may simultaneously act as receptor and ligand.

The term “couple” as used herein refers to directly or indirectly linking two moieties in any manner. For example, the term may refer to the attachment of a targeting moiety, such as an agonist, antibody or a ligand, to an effector molecule, such as a drug, radiotherapeutic compound, chemotherapeutic compound or enzyme, or a carrier molecule. The linkage can be, for example, either by chemical or recombinant means. Chemical linkage may involve a reaction between the targeting moiety and the effector molecule to produce a covalent bond formed between the two molecules to form one molecule.

The term “induce” when used in the context of cell differentiation, relates to the activation, stimulation, enhancement, initiation and/or maintenance of a differentiation program in the cell. For example, where the cell is a cancer cell, inducing differentiation may relate to the activation, stimulation, enhancement, initiation and/or maintenance of the cellular mechanisms or processes necessary to promote the transition of the cancer cell from a proliferative (cancerous or semi-cancerous) state into a non-proliferative (non-cancerous or benign) state.

The term “deliver” as used herein refers to the transfer of a substance or molecule (such as a bioactive compound with therapeutic or diagnostic activity) to a physiological site, tissue, or cell. Suitable delivery methods include, but are not limited to, buccal, sublingual, rectal, topical, nasal, intramuscular (e.g. intramuscular injection), intradermal (e.g. intradermal injection), subcutaneous (e.g. subcutaneous injection), intravenous, intradermal, and mucosal delivery.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically −/−4% of the stated value, more typically −/−3% of the stated value, more typically, −/−2% of the stated value, even more typically −/−1% of the stated value, and even more typically −/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### Disclosure of Optional Embodiments

Exemplary, non-limiting embodiments of an agonist of CD137 ligand and methods of its use, will now be disclosed.

The agonist of CD137L may be a fragment of CD137 protein, a recombinant CD137 protein, a CD137 fusion protein, an anti-CD137L antibody or a fragment thereof, a peptide, a small molecule, an aptamer, or an antibody mimetic.

The term “peptide” typically refers to molecules of 2 to 40 amino acid residues in length. Exemplary peptides having CD137L agonistic activity may be randomly generated by any of the methods known in the art, carried in a peptide library (e.g. a phage display library), derived by digestion of proteins (e.g. digestion of CD137 protein), or by chemical peptide synthesis. “Peptides” include cyclic peptides.

Exemplary antibody mimetics include anticalin and affilin.

In one embodiment, the agonist of CD137L is a recombinant CD137 protein comprising the extracellular domain of CD137 fused to the Fc region of human IgG (CD137-Fc). Hence, fusion proteins incorporating the proteins (e.g, CD137) and peptides described above are contemplated in the present disclosure. For example, it is often advantageous to include one or more additional amino acid sequences which may contain secretory or leader sequences, pro-sequences, or sequences which aid in for instance detection, expression, separation or purification of the protein or to endow the protein with additional properties as desired such as higher protein stability, for example during recombinant production, or for instance to produce an immunomodulatory response. Examples of potential fusion partners include epitope tags (short peptide sequences for which a specific antibody is available), polyethylene glycol, beta-galactosidase, luciferase, a polyhistidine tag, glutathione S transferase (GST), a secretion signal peptide and a label, which may be, for instance, bioactive, radioactive, enzymatic or fluorescent, or an antibody.

The fusion protein may be made using standard cloning and molecular biology methods known in the art. For example, a gene encoding a protein or peptide (e.g. a gene encoding CD137) can be amplified by polymerase chain reaction (PCR) and ligated to DNA coding for any of the above-described additional sequences to form a DNA molecule that encodes the fusion protein. The DNA molecule encoding the fusion protein can be cloned into any suitable vector, for example a plasmid vector. The vector may comprise a multiple cloning site into which the DNA molecule encoding the fusion protein can be easily inserted. The vector may also contain a selectable marker, such as an antibiotic resistance gene, to facilitate identification and isolation of bacteria transformed with the vector.

The recombinant CD137 protein as disclosed herein may be prepared using the above techniques based on sequence information that is available on public databases such as for example, Uni-Prot, Genbank, EMBL, and the like, which are all known to the person skilled in the art. For example, the sequence of the human CD137 may be extracted from Uni-Prot as shown in FIG. 14 (SEQ ID NO: 1). The corresponding nucleic acid sequence may be one as shown in FIG. 15 (SEQ ID NO: 2). Information pertaining to the various domains of CD137 are also known from such databases, for example, the extracellular domain spans from amino acid positions 24 to 186 of the human CD137 (SEQ ID NO: 1), the transmembrane domain spans from amino acid positions 187 to 213 of SEQ ID NO: 1, and the cytoplasmic domain spans from amino acid positions 214 to 255 of SEQ ID NO: 1. Amino acid and nucleic acid sequences for the Fc region of human IgG are likewise available in the public databases known to the person skilled in the art, for example as shown in FIG. 16 (SEQ ID NO: 3).

In one embodiment, the agonist of CD137L is an anti-CD137L antibody or fragment thereof. In one embodiment, the anti-CD137L antibody or fragment thereof comprises an agonistic anti-CD137L antibody or a fragment thereof.

The agonist of CD137L may be immobilized on a particle to facilitate crosslinking of CD137L and also delivery of the agonist to the target physiological site, tissue, or cell where it asserts its biological effect.

The agonist may be immobilized on a microparticle or a nanoparticle. Exemplary particles suitable for use include beads and microspheres. Particles can be made of a variety of materials including polymers (e.g. polystyrene), glass and ceramics. Other exemplary particles include magnetic, particles. Particles may be spherical beads having a diameter of at least 1 and not more than 10 microns or at least 2 and not more than 6 microns in diameter (e.g. about 3 microns in diameter).

According to some embodiments, the particles may have a diameter of less than 100 μm (e.g 1 μm).

Various protein immobilization methods are available for immobilizing protein on different kinds of materials for example, chemical activation, entrapment and crosslinking. Protein immobilization may be achieved through chemical covalent bonding or physical adsorption (e.g. non-covalent bonding) of, for example, glutaric dialdehyde or activated esters or by using photoactivatable crosslinkers, such as NHS-diarzirine.

In one embodiment, the agonist of CD137L is coupled to an agent selected from the group consisting of cytotoxic drugs, radiotherapeutics, chemotherapeutics, enzymes, and a carrier molecule. Advantageously, this enables delivery of such agents into the cell via internalization of the CD137L upon binding of the agonist to the CD137L. Exemplary cytotoxic drugs include many chemotherapeutic agents that are used in the treatment of cancers, including alkylating agents, antimetabolites, and toxins, e.g. vincristine, actinomycin, cisplatin, taxanes, paclitaxel, protaxel, dexamethasone, statins, sirolimus, and tacrolimus. Exemplary radiotherapeutics include radionucleotides, radioisotope labeled monoclonal antibodies, and other radioisotope labeled tumor homing agents and metabolites exhibiting specific preference for tumors. Radionuclides such as Re-188, Tc-99m, 1-131 and other iodine isotopes, F-18, C-11, O-15, In-1 11 may be used.

The agonist may also be coupled to a detectable agent, for example for use as a diagnostic tool. A “detectable agent” or “detectable label” refers to a reporter molecule or enzyme that is capable of generating a measurable signal and is covalently or non-covalently linked to the agonist. The detectable agent may be for example, a fluorescent agent, a luminescent label, an enzyme or a colorimetric label.

In one embodiment, the agonist of CD137L is for use in treating cancer. The method of treating cancer as disclosed herein comprises administering an agonist of CD137 ligand as defined herein to a subject in need thereof. The subject may be a human cancer patient.

The cancer that may be treated with the agonists as disclosed herein may be a myeloid malignancy, a lymphoid malignancy, or a multiple myeloma. The myeloid malignancy may be acute myeloid leukemia (AML), chronic myeloid leukemia (CML), acute promyelocytic leukemia (APL), chronic myelomonocytic leukemia (CMML), myelodysplastic syndrome, or juvenile myelomonocytic leukemia. The AML may be of any FAB subtype (based on the French-American-British classification), such as M0, M1, M2, M3, M4, M5, M5a, M5b, M6 or M7 subtype. The lymphoid malignancy may be chronic lymphocytic leukemia (CLL), acute lymphoblastic leukemia (ALL), B cell lymphoma, or mantle cell lymphoma (MCL). In one embodiment, the cancer is not APL or AML subtype M3.

The therapeutically effective dose level of the agonist for any particular patient will depend upon a variety of factors including: the disorder being treated and the severity of the disorder; activity of the agonist employed; the composition employed; the age, body weight, general health, sex and diet of the patient; the time of administration; the route of administration; the rate of sequestration of the agonist; the duration of the treatment; drugs used in combination or coincidental with the treatment, together with other related factors well known in medicine.

One skilled in the art would be able, by routine experimentation, to determine an effective, non-toxic amount of agonist which would be required to treat the applicable diseases.

Typically, in therapeutic applications, the treatment would be for the duration of the disease state.

Further, it will be apparent to one of ordinary skill in the art that the optimal quantity and spacing of individual dosages will be determined by the nature and extent of the disease state being treated; the form, route and site of administration; and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

Also disclosed is the use of the agonist of CD137L as defined herein in the manufacture of a medicament for treating cancer.

In general, suitable medicament compositions may be prepared according to methods which are known to those of ordinary skill in the art and accordingly may include a pharmaceutically acceptable carrier, diluent and/or adjuvant. Examples of pharmaceutically acceptable carriers or diluents are demineralised or distilled water; saline solution; vegetable based oils; silicone oils; volatile silicones; mineral oils; cellulose derivatives; lower polyalkylene glycols or lower alkylene glycols; polyvinylpyrolidone; agar; gum tragacanth or gum acacia, and petroleum jelly. For administration as an injectable solution or suspension, non-toxic parenterally acceptable diluents or carriers can include Ringer's solution, isotonic saline, phosphate buffered saline, ethanol and 1,2 propylene glycol. Suspensions for oral administration may further comprise dispersing agents and/or suspending agents. Solid forms for oral administration may contain binders, sweeteners, disintegrating agents, diluents, flavourings, coating agents, preservatives, lubricants and/or time delay agents. Adjuvants typically include emollients, emulsifiers, thickening agents, preservatives, bactericides and buffering agents. Methods for preparation of such compositions are apparent to those skilled in the art, and are described in more detail in, for example, Remington's Pharmaceutical Science, 15th ed., Mack Publishing Company, Easton, Pa., hereby incorporated by reference herein.

The CD137L agonists as defined herein may be used for inducing differentiation in cancer cells. The induction may be carried out in vivo, in vitro or ex vivo.

The CD137L agonists as defined herein may also be used for delivering a therapeutic or diagnostic agent to a cell in need thereof, comprising coupling an agonist of CD137L as defined herein to an agent selected from the group consisting of cytotoxic drugs, radiotherapeutics, chemotherapeutics, enzymes, and a carrier molecule. Exemplary cytotoxic drugs, radiotherapeutics and chemotherapeutics have been discussed above. Delivery may be via buccal, sublingual, rectal, topical, nasal, intramuscular (e.g. intramuscular injection), intradermal (e.g. intradermal injection), subcutaneous (e.g. subcutaneous injection), intravenous, intradermal, and mucosal routes.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1. is a series of histograms that show CD137L signaling induces immunophenotypical changes in AML cells consistent with differentiation. AML cells were cultured for 7 days in dishes coated with either immobilized Fc or CD137-Fc protein. Cells were then harvested and expression of surface markers was determined by immunostaining followed by flow cytometry. Depicted are histograms from two representative samples: (A) Sample 4, M2 subtype and (B) Sample 20, monocytic cells of unknown subtype. The open profile shows isotype controls while the solid and cross-hatched profiles show expression of the relevant surface marker by Fc- and CD137-Fc-treated cells, respectively. Numbers indicate the percentages of cells expressing the surface marker and the median fluorescence intensities (MFI).

FIG. 2. is a series of graphs that show CD137L signaling induces secretion of cytokines by AML cells. AML cells were cultured for 7 days in dishes coated with either immobilized Fc or CD137-Fc protein. Supernatants were then harvested and concentrations of cytokines were determined by ELISA. Depicted are means±standard deviations of triplicate measurements from one representative sample, Sample 19. (*P<0.05).

FIG. 3. is a series of photographs that show CD137L signaling induces adherence and morphological changes in AML cells. AML cells were cultured for 7 days in dishes, coated with either immobilized Fc or CD137-Fc protein. Photographs of cell morphologies from 3 different AML samples were taken on day 7 by bright-field light microscopy at a magnification of 40×.

FIG. 4. is a series of histograms that show CD137L signaling induces up-regulation of CD83 and decreases phagocytosis in monocytic AML cells. AML cells were cultured for 7 days in dishes coated with either immobilized Fc or CD137-Fc protein. Cells were then harvested and analysed. CD83 expression was determined by immunostaining followed by flow cytometry, as shown in (A). Depicted are histograms from three AML samples. The open histograms show the isotype controls while the solid histograms show CD83 expression. Numbers indicate percentages of cells expressing CD83. Phagocytosis was determined by incubation and uptake of fluorescent latex beads 45 minutes before analysis by flow cytometry, as shown in (B). Depicted are histograms from two AML samples. The open histograms show cell autofluorescence while the solid histograms show phagocytic cells. Numbers indicate percentages of phagocytic cells and MFI.

FIG. 5. shows CD137-Fc-treated AML cells enhance allogeneic T cell activation. AML cells were cultured for days in dishes coated with either immobilized Fc or CD137-Fc protein. The cells were then harvested and co-cultured with CFSE-labelled allogeneic T cells at a ratio of 1:10. The co-cultures were sub-optimally activated with an agonistic anti-CD3 antibody (clone OKT-3). After 5 days, the T cells were harvested and analysed. This experiment was performed using three AML samples (numbers 18, 19 and 20) with comparable results. Depicted are the results from a representative sample, Sample 18. (A) T cell proliferation was determined by CFSE dilution and flow cytometry. Areas marked by horizontal bars indicate proliferative cells. Numbers indicate percentages of proliferating T cells, and MFI of the total cell population. (B) The concentrations of several cytokines in co-culture supernatants were determined by ELISA. Depicted are means standard deviations of triplicate measurements. (*P<0.05).

FIG. 6. shows CD137-Fc-treated AML cells demonstrate reduced proliferation. CFSE-labelled AML cells were cultured for 7 days in dishes coated with either immobilized Fc or CD137-Fc protein, after which 50 ng/ml IFN-γand 10 ng/ml GM-CSF were added to induce proliferation. After 4 days, cells were harvested and proliferation was quantified. Depicted are results from one representative sample, Sample 21. (A) CFSE dilution. Areas marked by horizontal bars indicate proliferative cells. Numbers indicate percentages of proliferating cells and MFI of total cell population. (B) ³H-thymidine incorporation. Depicted are means±standard deviations of triplicate measurements. (*P<0.05).

FIG. 7. is a graph showing increased invasiveness of CD137-Fc-treated AML cells. AML cells were cultured for 7 days in dishes coated with either immobilized Fc or CD137-Fc protein. Cells were then harvested and placed into Matrigel-coated Transwell chambers suspended over a lower chamber containing 10% FBS as a chemoattractant. After 24 h, cells that had migrated across the Transwell membrane into the lower chamber were counted. Depicted are means±standard deviations of triplicate measurements from one representative sample, Sample 21. (*P<0.05).

FIG. 8. shows side scatter characteristics and CD45 expression of AML cells from representative samples. (A) Sample 2, Ml; (B) Sample 8, M2; and (C) Sample 17, M5b.

FIG. 9. shows indirect detection of CD137L via binding of CD137-Fc. AML cells were placed into culture dishes coated with either immobilized Fc or CD137-Fc protein for one hour. Levels of bound CD137-Fc were subsequently determined by immunostaining with anti-CD137 antibody and flow cytometry. Unspecific binding was prevented by addition of Fc receptor blockers.

FIG. 10. shows the efficacy of a first formulation of recombinant CD137 protein. In (A), CD137-Fc was conjugated to the surface membrane of red blood cells (RBC). Freshly isolated red blood cells were biotinylated, bonded with avidin, then conjugated to biotinylated CD137-Fc. The presence of CD137-Fc on the red blood cells was determined by immunostaining followed by flow cytometry. Open histograms show staining with the isotype controls while the hatched histograms show staining for CD137. Numbers indicate percentage of cells positive for CD137. (B) shows RBC-CD137-Fc induces IL-8 secretion by monocytes. Monocytes were cultured for 5 days with RBCs conjugated with CD137-Fc or control RBCs conjugated only with biotin, or in dishes coated with immobilized CD137-Fc or Fc protein. The concentration of IL-8 in culture supernatants was determined by ELISA. Depicted are means±standard deviations of triplicate measurements. (*P<0.05).

FIG. 11. shows the efficacy of a second formulation of recombinant CD137 protein. L428 cells that were transfected with an empty vector (L428-control) or with a CD137 expression vector (L428-CD137) were stained with an isotype (white histogram) or an anti-CD137 antibody (grey histogram). Numbers indicate the percentages of positive cells.

FIG. 12. shows CD137-transfected L428 cells co-cultured with monocytes induce cytokine secretion. L428-control or L428-CD137 cells were co-cultured at indicated ratios with monocytes. Numbers depicted on the x-axis represent the monocyte:L428 ratio. After 24 h, the cytokine concentrations in co-culture supernatants were determined by ELISA. Depicted are means±standard deviations of triplicate measurements. (*P<0.05).

FIG. 13. shows CD137 protein induces immunophenotypic changes consistent with myeloid differentiation in a mouse model of AML. Bone marrow cells from CbfJ3 knockout mice were cultured for 5 days in dishes coated with immobilized Fc or CD137-Fc protein. Cells were then harvested and expression of surface markers was determined by immunostaining followed by flow cytometry. Depicted are histograms showing surface marker expression. The open profile shows the isotype control while the hatched profiles show expression of the relevant surface marker. Numbers indicate the percentages of cells expressing the surface marker and the MFI. This data is representative of two independent experiments with comparable results.

FIG. 14. shows the amino acid sequence of a human CD137 (SEQ ID NO: 1).

FIG. 15. shows the nucleic acid sequence encoding a human CD137 (SEQ ID NO: 2).

FIG. 16. shows the nucleic acid sequence encoding the Fc region of human IgG1 (SEQ ID NO: 3).

### EXAMPLES

Non-limiting examples of the invention will be further described in greater detail by reference to specific Examples, which should not be construed as in any way limiting the scope of the invention.

### Example 1

This example shows that recombinant CD137 protein, which crosslinks CD137L and initiates reverse CD137L signaling in myeloid cells, induces morphological changes (in adherence and spreading), loss of progenitor markers (CD117), expression of maturation markers (CD11b, CD13) and secretion of cytokines that are indicative of myeloid differentiation. Under the influence of CD137L signaling, AML cells acquired expression of co-stimulatory molecules (CD80, CD86, CD40), the dendritic cell marker CD83, and dendritic cell activities, enabling them to stimulate T cells. CD137L signaling induced differentiation in 70% (14 of 20) of AML samples, irrespective of FAB classification and the level of CD137L expression. However, the type of response varied with the AML subtype and patient sample. In summary, this example demonstrates that CD137L signaling induces differentiation in malignant cells of AML patients, and suggests treatment with recombinant CD137 protein as a therapeutic approach for AML.

### Methods

### Recombinant Proteins

CD137-Fc protein was purified from the supernatants of stable transfected CHO cells by protein G sepharose, as described previously (Schwarz, et al 1996). Human IgG1 Fc protein was purchased from Chemicon International.

### Patient Samples

AML patient samples were collected at the Singapore General Hospital and the National University Cancer Institute, Singapore under protocols approved by the institutional review boards. Peripheral blood or bone marrow samples were taken from patients with AML at the time of diagnosis following written informed consent. Mononuclear cells were isolated by density gradient centrifugation using Ficoll-Paque (1.077 g/dl, GE Life Sciences). Diagnosis was established by cytological criteria based on the FAB classification. Isolated mononuclear cells were cultured immediately. 21 samples were analysed for this study.

### Cell Culture

Mononuclear AML cells were cultured at a density of 2×10⁶ cells/ml in polystyrene culture dishes (Becton Dickinson) coated with 10 μg/ml Fc or 10 μg/ml CD137-Fc protein, in RPMI-1640 supplemented with 10% FES, 50 μg/ml streptomycin and 50 IU/ml penicillin.

### Immunophenotypic Analysis by Flow Cytometry

Flow cytometric analysis was performed before and after 7 days of Fc- or CD137-Fc-treatment to determine surface marker expression. Cells were stained with antibodies in PBS containing 0.5% BSA and 0.1% sodium azide (FACS buffer) at 4° C. for 30 min. Non-specific staining was controlled by labelling with isotype-matched antibodies. Cells were then washed and resuspended in 300 μl FACS buffer. Flow cytometry was performed on a CyAn FACS machine (Dako).

Antibodies directed against the following targets were used: CD117-PE (clone 104D2), CD34-APC (clone 4H11), CD14-APC (clone 61D3), CD36-FITC (clone NL07), CD11b-PE (clone ICRF44), CD13-APC (clone WM15), CD80-PE (clone 2D10.4), CD86-PE (clone IT2.2), CD40-PE (clone FC3), CD83-APC (clone HB15E), HLA-DR-APC (clone LN3), CD45-PE-Cy7 (clone HI30) (all from eBioscience), CD137L-PE (clone 5F4) (Biolegend) and CD137-PE (BD Biosciences). Forward- and side-scatter characteristics in combination with CD45 expression were used to identify viable AML cells of interest.

### Assessment of Phagocytosis

Phagocytosis assay was performed on Fc- and CD137-Fc-treated cells after 7 days of culture. Cells were incubated with Yellow-Green fluorescent FluoroSphere microspheres (Invitrogen) at a concentration of 50 beads per cell at 37° C. for 45 min. Phagocytosis was stopped by the addition of ice-cold PBS, cells were washed and treated with trypsin for 5 min to detach any surface-adherent beads. Cells were washed again and resuspended in 300 μl PBS. Flow cytometry was performed to determine percentage of cells that had phagocytosed beads.

### Cell Morphology

Morphology of Fc- and CD137-Fc-treated cells was evaluated after 7 days of culture. Cell smears were obtained by air-drying of cell suspensions onto a glass slide followed by staining with a modified wright-giemsa stain. Bright-field light microscopy photographs were taken using the Zeiss Axiovert 40 inverted microscope (Zeiss) and Canon. Powershot G6 digital camera.

### Detection of Cytokine Secretion by ELISA

The concentrations of IL-8, TNF-α, MCP-1, M-CSF, IL-10, IL-12p70, IL-23, IFN-γ, IL-13 and IL-17 in culture supernatants were measured using the respective R&D DuoSet ELISA kits (R&D Systems) according to the manufacturer's instructions. Samples were assayed in triplicate within each experiment.

### Allogeneic Mixed Lymphocyte Reaction

The ability of pre-treated AML cells to stimulate T cells was assessed in an allogeneic mixed lymphocyte reaction. Peripheral blood mononuclear cells (PBMC) were isolated from buffy coat of healthy donors by density gradient centrifugation using Ficoll-Paque (1.077g/dl, GE Life Sciences), followed by positive-selection of CD3⁺ T cells using CD3 Microbeads (Miltenyi Biotec) according to the manufacturer's instructions. Fc- and CD137-Fc-treated AML cells were harvested after 7 days of culture and subsequently co-cultured with CD3⁺ T cells at a ratio of 1:10 and density of 10⁶ cells/ml. Co-cultures were sub-optimally activated with 1.1 ng/ml of agonistic anti-CD3 antibody, OKT-3, and maintained in RPMI-1640 supplemented with 10% FBS, 50 ug/ml streptomycin and 50 IU/ml penicillin for 5 days. To assess T cell proliferation, T cells were stained with CFSE (Invitrogen) according to the manufacturer's instructions prior to co-culture. CFSE dilution was assessed by flow cytometry. Supernatants were collected for detection of cytokine secretion by ELISA.

### Assessment of AML Proliferation

AML cells were stained with CFSE (Invitrogen) according to the manufacturer's instructions prior to culture. After 7 days of Fc- or CD137-Fc-treatment, proliferation was induced by addition of 50 ng/ml IFN-γ and 10 ng/ml GM-CSF (Peprotech) to the culture media. After an additional 4 days, the cells were harvested and CFSE dilution was assessed by flow cytometry. Alternatively, cells were pulsed with 0.5 μCi of ³H-thymidine (Perkin Elmer) for the last 18 h of the culture period. The cells were then harvested onto a Packard Unifilter Plate using a MicroMate 196 Cell Harvester and analysed for incorporation of ³H-thymidine using TopCount Microplate Scintillation Counter (Perkin Elmer). Each condition was assayed in triplicate.

### Transwell Migration Assay

The effect of CD137 on in vitro migration of AML cells was assayed using Matrigel-coated Boyden chambers. Cells were harvested after 7 days of Fc- or CD137-Fc treatment, washed twice and starved for 4 h at 4° C. in serum-free RPM-1640 media. 24-well Transwell cell culture chambers with 5.0 μm-pore polycarbonate inserts (Corning Incorporated) were coated with 100 μl Matrigel (BD Biosciences) for 2 h at 37° C. and then washed. 100 μl cell suspension were then placed into the Matrigel-coated chambers at a density of 6×10⁵ cells/ml in serum-free RPM-1640 media. These chambers were then placed into 24-well culture dishes containing 500 μl RPM-1640 supplemented with 10% FBS as a chemoattractant and incubated for 24 h at 37° C. After the incubation period, cells that had migrated into the bottom chamber were stained with trypan blue and counted under a light microscope.

### Statistics

Where relevant, statistical significance was determined by a two-tailed unpaired Student's t-test. P-values of <0.05 were considered significant.

### Results

Mononuclear cells freshly isolated from either bone marrow or peripheral blood of AML patients were placed immediately into culture dishes coated with a recombinant protein consisting of the extracellular domain of CD137 fused to the Fc region of human IgG (CD137-Fc). Cells cultured in dishes coated only with the Fc protein were used as negative controls. After 7 days, the cells were analysed for evidence of differentiation.

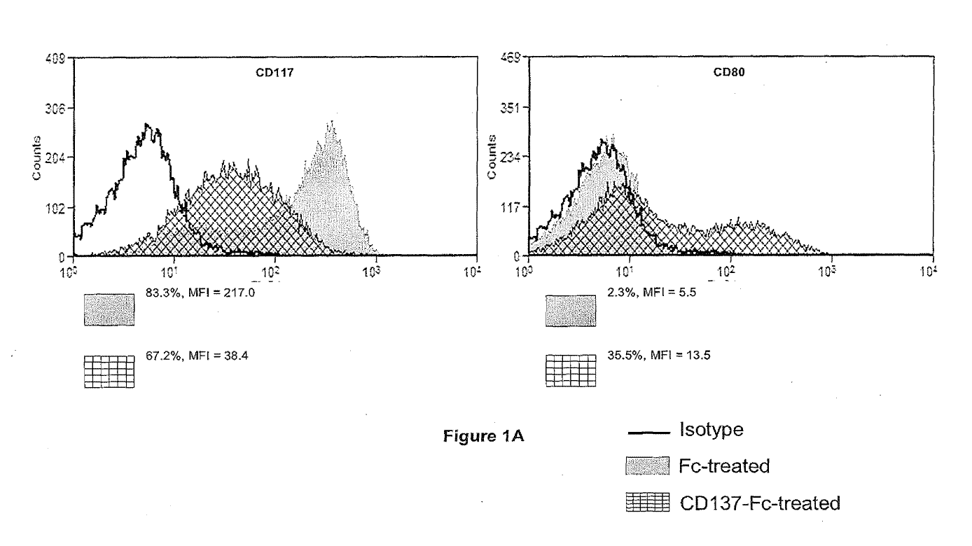

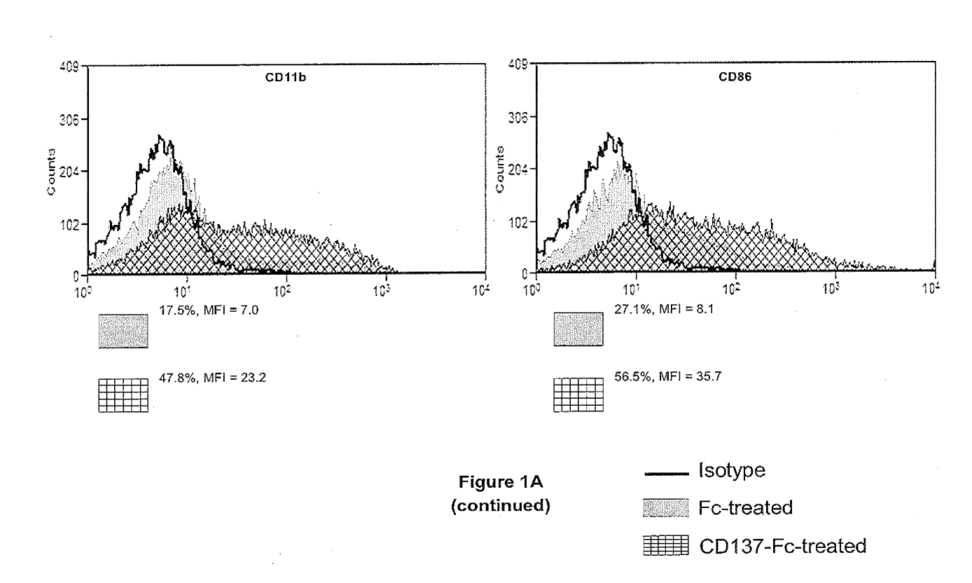

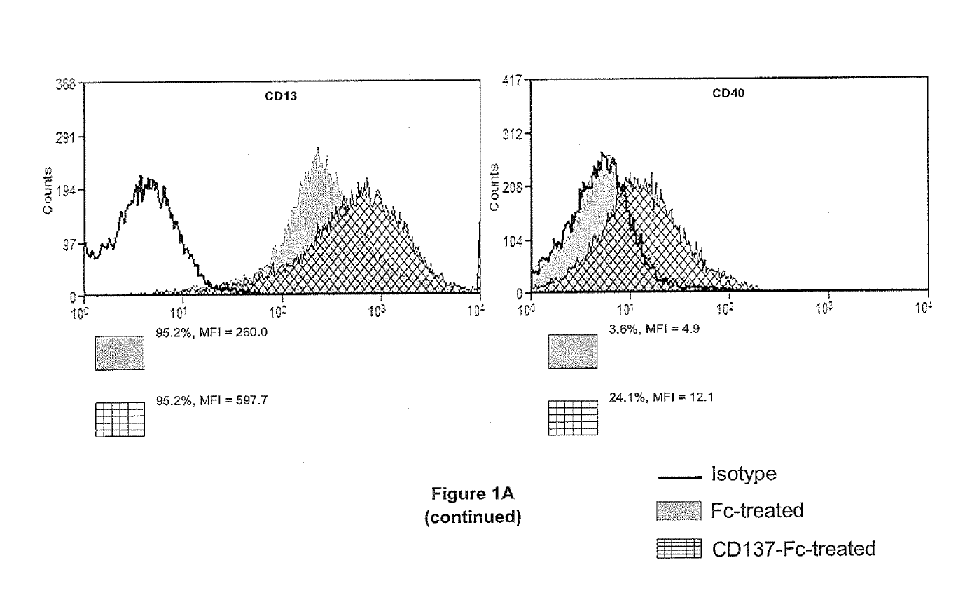

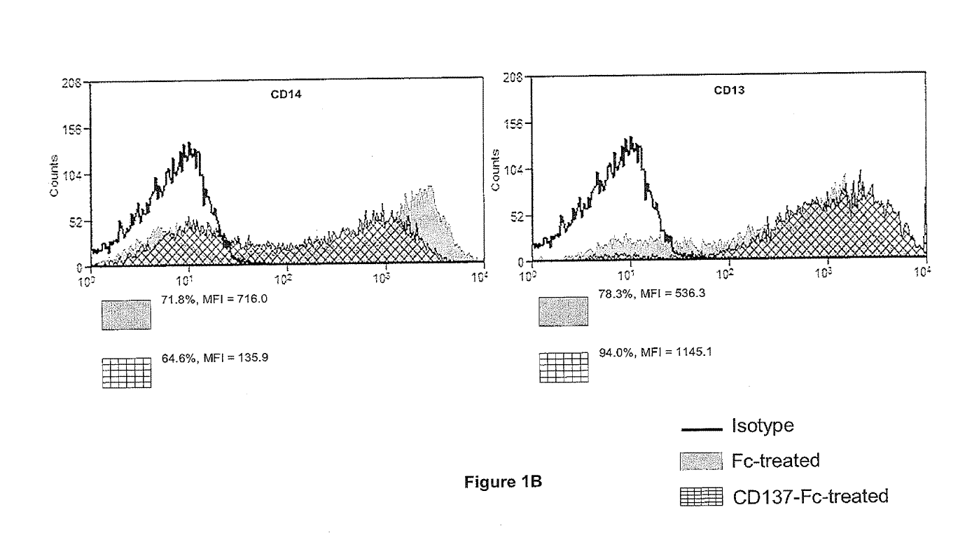

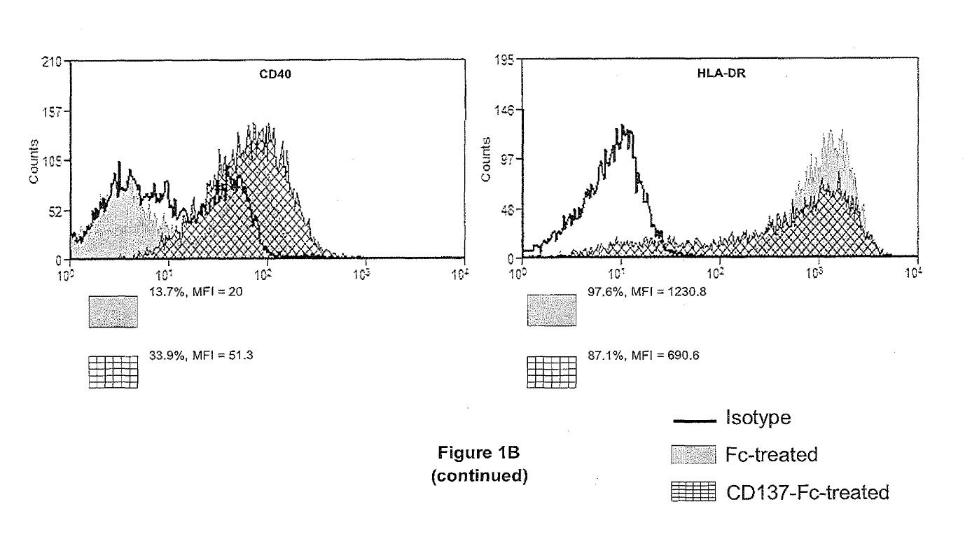

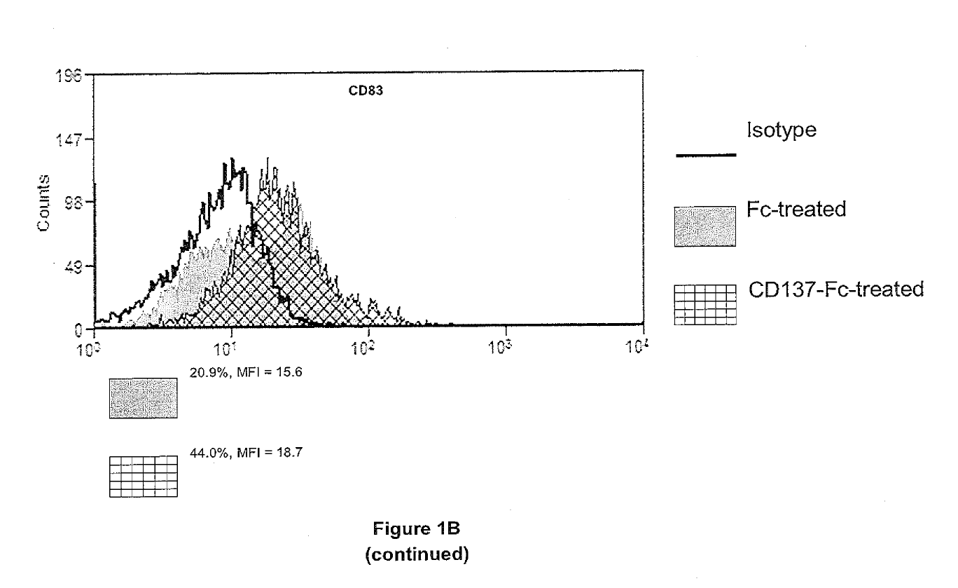

AML cells treated with recombinant CD137 protein acquired a more mature immunophenotype compared to control cells. In a representative sample, both the median fluorescence intensity (MFI) and percentage of cells expressing the hematopoietic progenitor cell marker CD117 were strongly decreased (FIG. 1A). This was accompanied by marked up-regulation of the myelomonocytic markers CD11b and CD13, and acquisition of the co-stimulatory molecules CD80, CD86 and CD40. A representative AML sample with a monocytic immunophenotype showed down-regulation of the monocyte marker CD14, and the MHC class II molecule, HLA-DR together with increased expression of CD13, CD40 and the dendritic cell marker CD83 (FIG. 1B). Treatment with the Fc control protein had minimal or no effect on the immunophenotype (samples 4, 6, 8, 15 and 18) when compared to untreated cells at the beginning of the study (not shown).

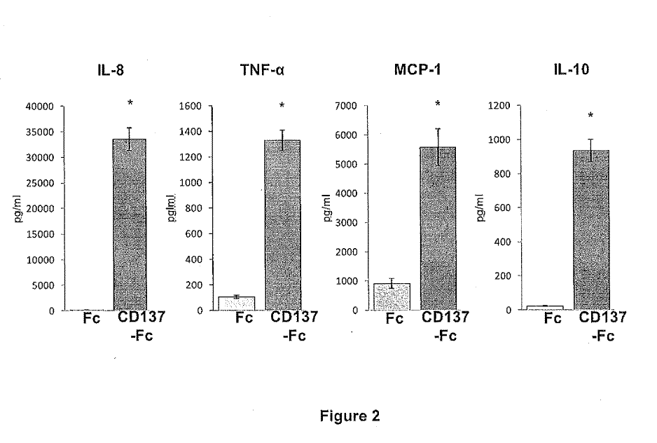

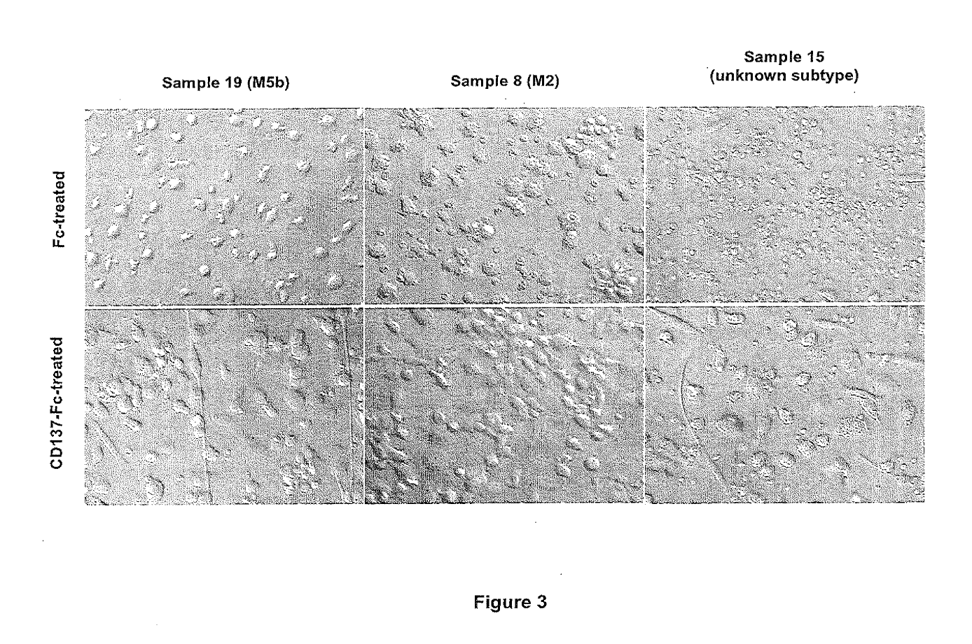

Treatment of the AML cells with recombinant CD137 protein strongly induced secretion of the pro-inflammatory cytokines IL-8, TNF-a and MCP-1, and the anti-inflammatory cytokine IL-10 (FIG. 2). IL-12 and IL-23, which play roles in T cell activation and polarization, could not be detected in any of the AML culture supernatants (data not shown). Several samples also acquired morphologies characteristic of dendritic cells (FIG. 3). The majority of these cells became adherent and adopted flattened, spindle-shaped morphologies with dendrite-like extensions.

Since similar changes are induced by CD137L signaling in myeloid or monocytic cells during normal myelopoiesis from healthy individuals (Langstein, et al 2000, Langstein, et al 1998, Langstein, et al 1999, Langstein and Schwarz 1999), these observations in AML cells indicate that they can also undergo some degree of differentiation to a more mature phenotype in response to treatment with CD137-Fc protein.

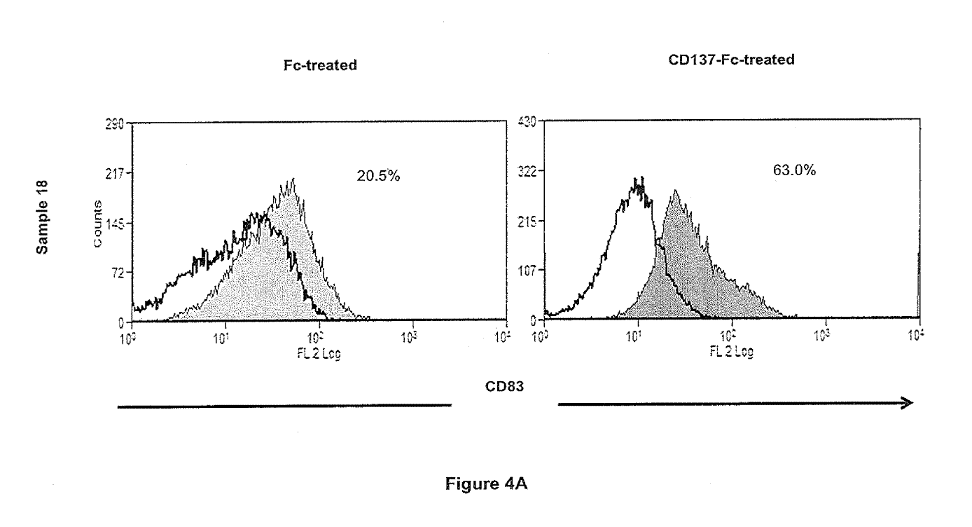

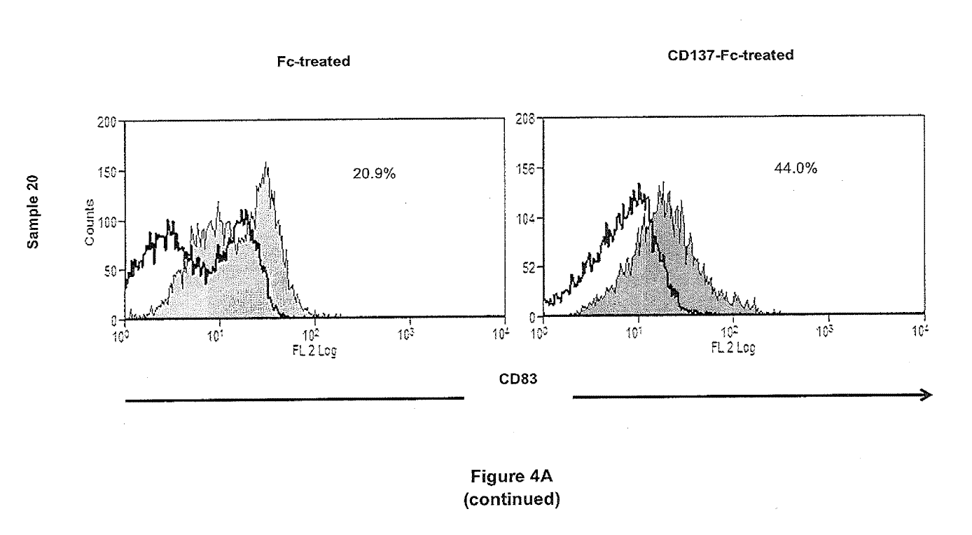

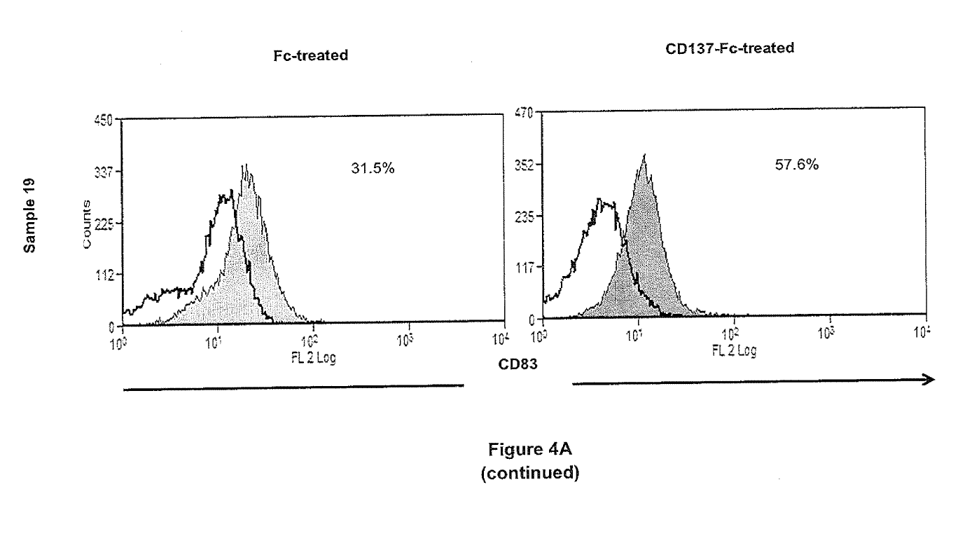

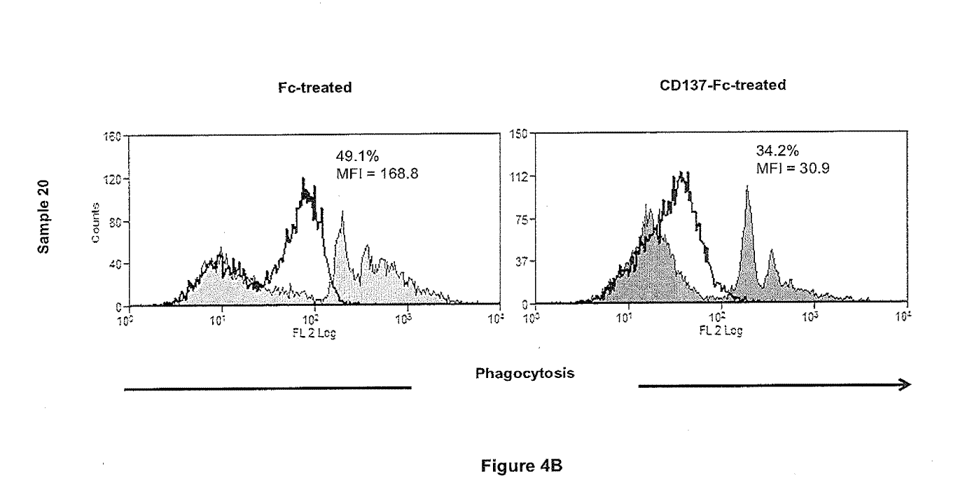

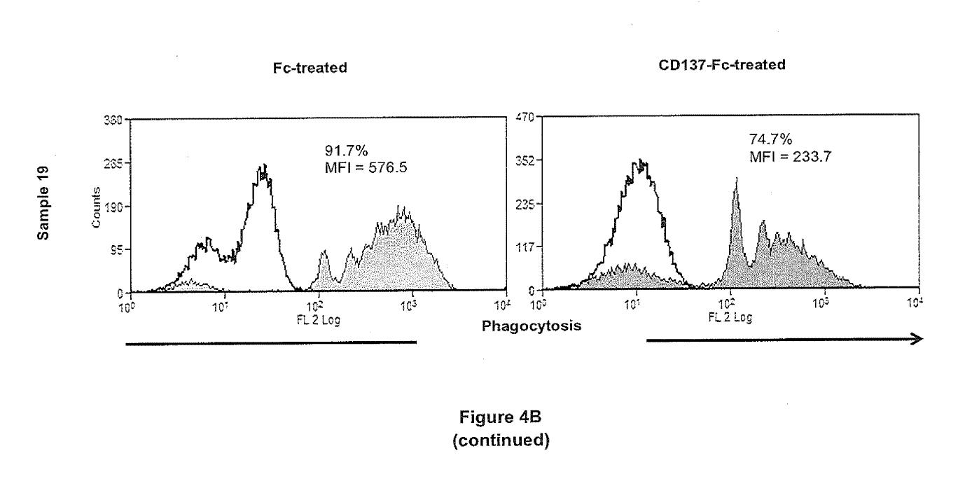

Since CD137L signaling induces DC differentiation of peripheral human monocytes (Ju, et al 2009, Kwajah and Schwarz 2010), and maturation of immature DC (Kim, et al 2002, Laderach, et al 2003, Lippert, et al 2008), whether AML cells would acquire DC-like properties upon exposure to recombinant CD137 protein was investigated. When monocytic AML cells mainly of the M5b subtype and characterized by high CD14 expression were treated with CD137 protein, the DC marker CD83 was observed to be up-regulated (FIG. 4A) while the phagocytic ability of these cells was decreased (FIG. 4B). These changes, which were observed only in monocytic AML samples, were consistent with differentiation of these cells into DC-like cells (Kwajah and Schwarz 2010).

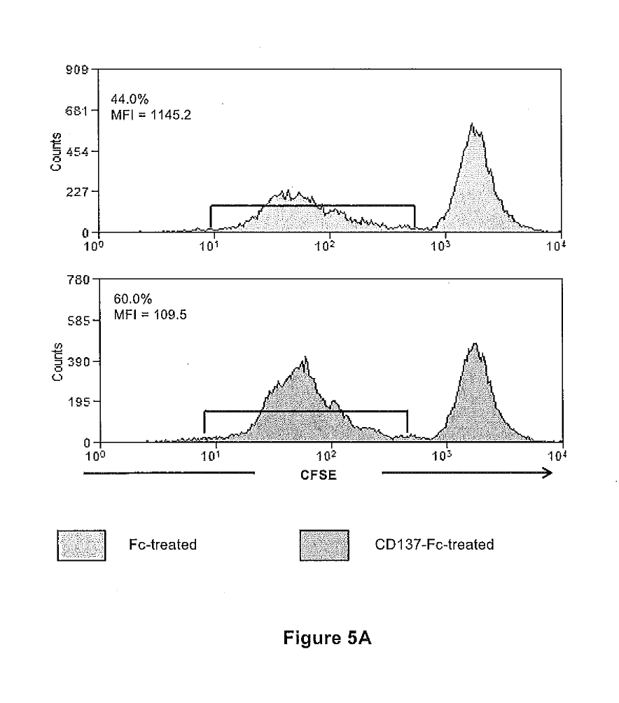

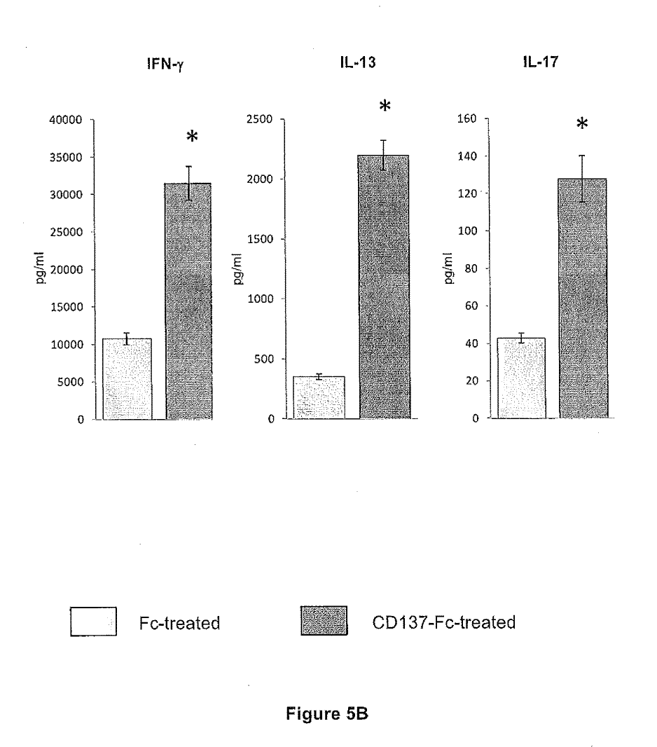

To determine whether the AML cells treated with CD137 possessed enhanced capacity to stimulate T cells, an allogeneic mixed lymphocyte reaction was performed. After 7 days of treatment with either CD137-Fc or Fc, the AML cells were harvested and co-cultured with CFSE-labeled allogeneic T cells at a ratio of 1:10. T cells stimulated with CD137-Fc-treated AML cells demonstrated higher proliferation compared to those stimulated with Fc-treated AML cells. 60 compared to 44% of the T cells had divided, and the MFI of CFSE had dropped from 1145 to 109 (FIG. 5A). Further, T cells that were co-cultured with the CD137-Fc-treated AML cells released more than 3-fold higher levels of the pro-inflammatory cytokines IFN-γ, IL-13 and IL-17 than the control cells (FIG. 5B). These results demonstrated that CD137-treated AML cells possess enhanced T cell-activating capacities.

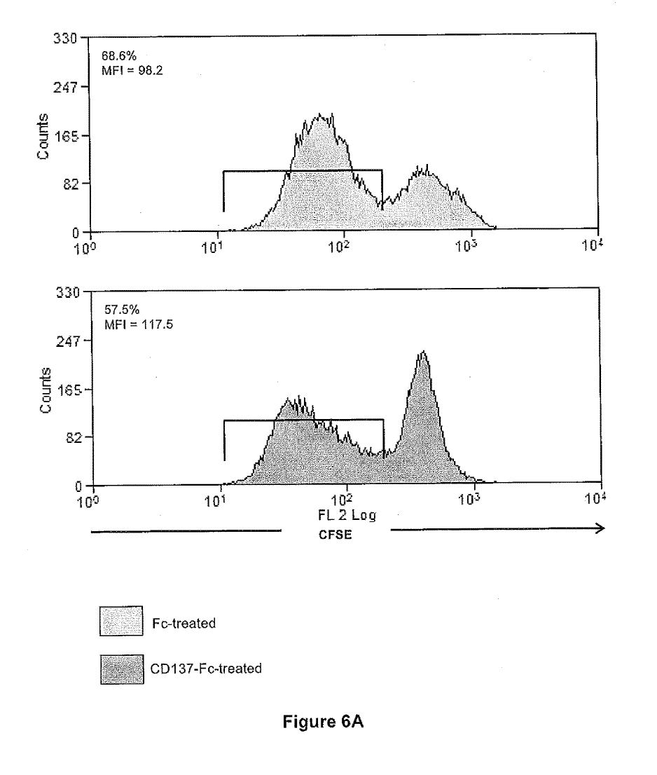

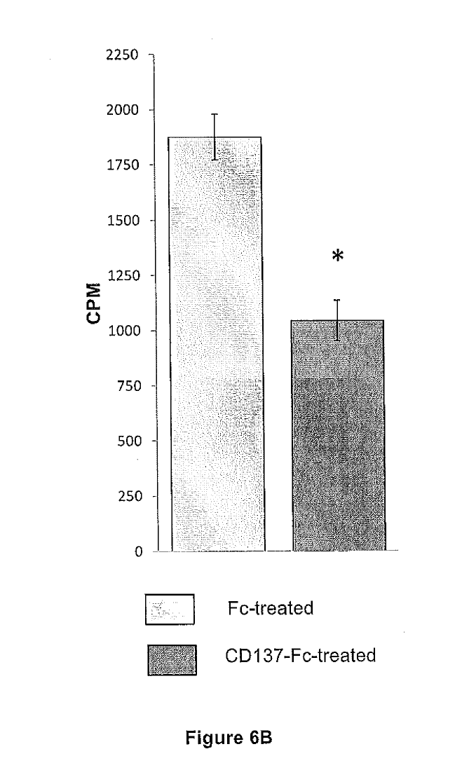

To determine whether the observed differentiation of the AML cells resulted in decreased proliferative capacity, a proliferation assay was performed. As the AML cells did not proliferate in vitro in the absence of growth factors, proliferation was induced by the addition of IFN-γ and GM-CSF, after 7 days of treatment with CD137-Fc or Fc protein. CD137-Fc-treated AML cells demonstrated a significantly reduced proliferation rate compared to the control cells (FIG. 6). This data shows that CD137-Fc-induced differentiation of AML cells is also accompanied by decreased proliferative capacity.

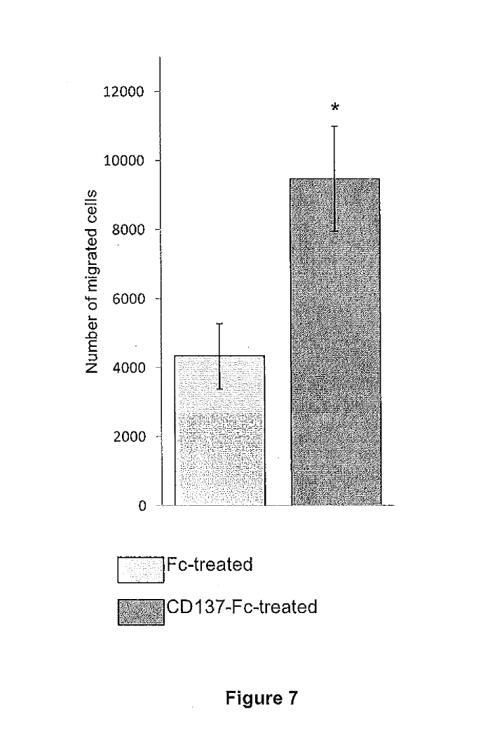

Consistent with the observation that CD137L signaling enhances migration of monocytes in vitro (Drenkard, et al. 2007), CD137-Fc-treated AML cells also possess enhanced migratory potential. After 7 days, a 2-fold greater number of CD137-Fc-treated AML cells was able to migrate across a porous membrane compared to Fc-treated control cells (FIG. 7).

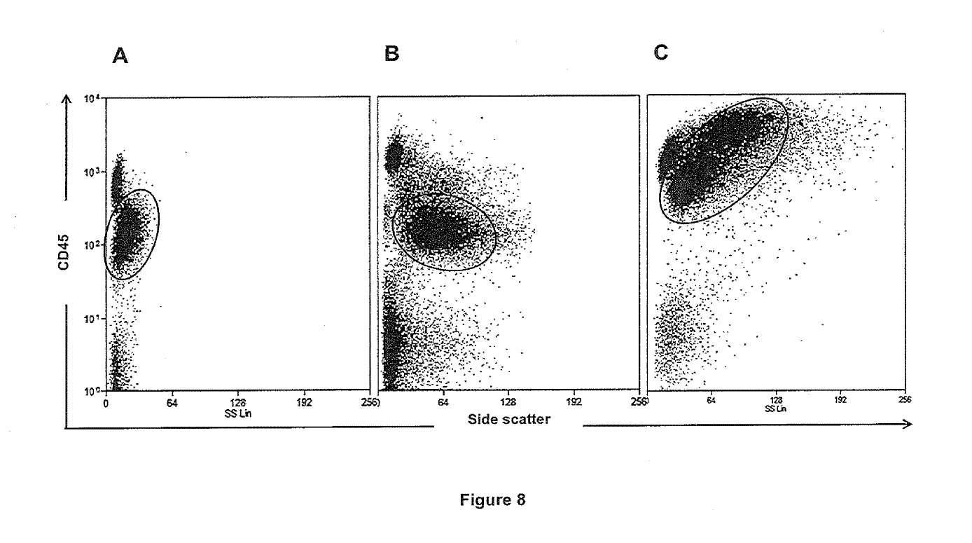

Since AML samples derived from a range of FAB subtypes with different stages of maturity were assessed for evidence of CD137 protein-induced differentiation, the changes observed varied considerably between individual samples. The samples were therefore grouped into three categories, based on the degree of maturity and the myeloid progenitor cell from which the AML originated. AML blasts with minimal or no maturation were characterized by low side scatter and moderate expression of the pan-leukocyte marker CD45 (FIG. 8A). These cells typically expressed high levels of the progenitor cell markers CD117 and CD34, did not express CD11b or CD14 and were of the MI or M2 subtype. Cells with a greater degree of maturation exhibited moderate side scatter and CD45 expression (FIG. 8B), co-expressed CD117 or CD34 with CD11b, and were of the M2 or M5a subtype. Monocytic leukemia cells had moderate to high side scatter and high CD45 expression (FIG. 8C), expressed CD14, and were of the M5b subtype.

Upon treatment with recombinant CD137 protein, AML cells with minimal or no maturation (Table 1) down-regulated CD117 or CD34, while acquiring expression of CD11b in every sample. CD13 and HLA-DR were variably up-regulated in several samples. The secretion of pro-inflammatory cytokines was markedly increased ranging from to 100-fold in all samples over that of the control treated cells.

TABLE 1
Fold change in MFI of surface markers and cytokine
concentrations for CD137-Fc-treated compared to Fc-
treated AML cells with minimal or no maturation.

| | | | |
|---|---|---|---|
| Sample 1 | Sample 4 | Sample 6 | Sample 7 |
| (M1) | (M2) | (n.a.) | (n.a.) |

Fold change in MFI of surface markers

| | | | | |
|---|---|---|---|---|
| CD117 | 1.3 | −5.7 | −2 | −2.6 |
| CD34 | −7.5 | −1.2 | 1.0 | −2 |
| CD14 | −1.1 | 1.1 | 1.0 | 7.2 |
| CD11b | 3.6 | 3.3 | 2.3 | 3.2 |
| CD13 | −1.4 | 2.3 | 3.0 | 3.6 |
| CD86 | −1.6 | 4.4 | −3 | 1.8 |
| CD40 | n.a. | 2.5 | 1.0 | −1.3 |
| HLA-DR | 14.5 | −1.1 | 3.3 | −1.2 |

Fold change in cytokine concentration

| | | | | |
|---|---|---|---|---|
| IL-8 | 2.3 | n.d. | 2.8 | 113 |
| TNF-α | 117 | 170 | n.d. | 2.0 |
| MCP-1 | 30 | n.d. | n.d. | 36 |
| M-CSF | 9 | 3.9 | 31 | n.d. |
| IL-10 | n.d. | 6.7 | 2.3 | 3.8 |

Underlined numbers indicate a greater than 1.8-fold change or marked increase in percentage of cells expressing the antigen.
Means ± standard deviations of triplicate measurements from each sample were used to calculate fold change in cytokine concentrations.
All values are significant (P < 0.05).
(n.a. means not available; n.d. means none detected).

Cells with some degree of maturation (Table 2) also down-regulated CD117 and CD34. Expression of CD11b and the monocyte marker, CD14, were profoundly enhanced in most of the samples, along with acquisition of the co-stimulatory molecules CD86 and CD40.

TABLE 2
Fold change in MFI of surface markers and cytokine concentrations for
CD137-Fc-treated as compared to Fc-treated AML cells with increased maturation.

| | Sample 8 | Sample 9 | Sample | Sample | Sample | Sample | Sample |
|---|---|---|---|---|---|---|---|
| | (M2) | (M2) | 12 (M2) | 13 (M5a) | 14 (n.a.) | 15 (n.a.) | 21 (n.a.) |
| | Fold change in MFI of surface markers | | | | | | |
| CD117 | −3.4 | −5.3 | −1.1 | −9.7 | −1.1 | −13 | −7.3 |
| CD34 | −3.3 | −2.9 | −2.0 | 1.0 | 1.0 | 1.0 | 1.0 |
| CD14 | −1.1 | 2.2 | −2.0 | 31.5 | 49.4 | 5.9 | 10.0 |
| CD11b | 9.0 | 12.0 | 2.9 | 3.9 | −1.8 | 4.1 | 38.0 |
| CD13 | −1.1 | 1.2 | 2.4 | 1.4 | 7.3 | 7.8 | 9.1 |
| CD86 | −1.4 | 2.2 | 1.3 | 1.2 | 2.8 | 6.5 | 4.2 |
| CD40 | −2.0 | 1.0 | 1.5 | 2.0 | 1.3 | 2.2 | — |
| HLA-DR | −2.1 | 1.0 | −1.4 | −1.4 | −1.8 | 7.8 | — |
| | Fold change in cytokine concentration | | | | | | |
| IL-8 | 1.0 | 2.4 | 4.0 | 2.2 | 32 | 3.0 | — |
| TNF-α | −2.0 | 2.4 | 25 | 2.3 | 5.0 | n.d. | — |
| MCP-1 | n.d. | n.d. | n.d. | n.d. | 4.0 | −10 | — |
| M-CSF | 2.4 | 4.5 | n.d. | n.d. | n.d. | −3.0 | — |
| IL-10 | −3.6 | 1.3 | 1.6 | 2.2 | 63 | n.d. | — |

Underlined numbers indicate a greater than 1.8-fold change or marked increase in percentage of cells expressing the surface marker. Means ± standard deviations of triplicate measurements from each sample were used to calculate fold change in cytokine concentrations. All values are significant (P < 0.05). (n.a. means not available; n.d. means none detected).

In CD14-positive monocytic AML cells (Table 3), changes in expression of progenitor cell markers and co-stimulatory molecules were less pronounced. However, expression of CD14 was decreased in 3 of 4 samples and HLA-DR was decreased in two samples. Up-regulation of CD83 was observed (FIG. 4a) with concomitant suppression of the phagocytic ability of these cells (FIG. 4B). With the exception of Samples 8, 15 and 17, treatment with CD137 protein enhanced secretion of at least two cytokines in every sample.

TABLE 3
Fold change in MFI of surface markers and cytokine
concentrations for CD137-Fc-treated as compared
to Fc-treated monocytic AML cells.

| | | | |
|---|---|---|---|
| Sample 17 | Sample 18 | Sample 19 | Sample 20 |
| (M5b) | (M5b) | (M5b) | (n.a.) |

Fold change in MFI of surface markers

| | | | | |
|---|---|---|---|---|
| CD117 | 1.0 | −1.7 | −2.3 | 1.3 |
| CD34 | −1.9 | −1.5 | −1.5 | −1.2 |
| CD14 | 2.2 | −2.4 | −4.7 | −5.3 |
| CD11b | 2.8 | 1.2 | −9.4 | 2.0 |
| CD13 | 1.1 | 4.4 | 4.2 | 2.1 |
| CD86 | n.a. | 1.8 | −1.2 | 1.4 |
| CD40 | n.a. | 1.0 | 1.3 | 2.5 |
| HLA-DR | n.a. | −1.3 | −5.7 | −1.8 |

Fold change in cytokine concentration

| | | | | |
|---|---|---|---|---|
| IL-8 | 1.0 | 42 | 254 | 3.0 |
| TNF-α | −3.2 | n.d. | 13 | n.d. |
| MCP-1 | n.d. | n.d. | 6.0 | n.d. |
| M-CSF | n.d. | 10 | n.d. | 14 |
| IL-10 | −1.4 | 3.2 | 42 | n.d. |

Underlined numbers indicate a greater than 1.8-fold change or marked increase in percentage of cells expressing the surface marker.
Means ± standard deviations of triplicate measurements from each sample were used to calculate fold change in cytokine concentrations.
All values are significant (P < 0.05).
(n.a. means not available; n.d. means none detected).

Taken together, this data show that AML cells upon induction of CD137L-signaling by recombinant CD137 protein exhibit different but recognizable patterns of immunophenotypic and functional changes, depending on the stage of maturation or type of cell from which the leukemic cell was derived from, and these changes are consistent with progressing myeloid differentiation.

Twenty freshly isolated AML samples of various subtypes were tested in this study (Table 4). Of these, 14 (70%) demonstrated evidence of having undergone some degree of differentiation in response to treatment with recombinant CD137 protein. In 6 samples, no changes in immunophenotype, cytokine release, morphology or cell functions were observed (data not shown). These samples were deemed unresponsive and resistant to CD137 protein-induced differentiation. There did not appear to be a correlation between sensitivity to differentiation and cytogenetic characteristics or FAB subtype, as samples from each subtype were able to differentiate.

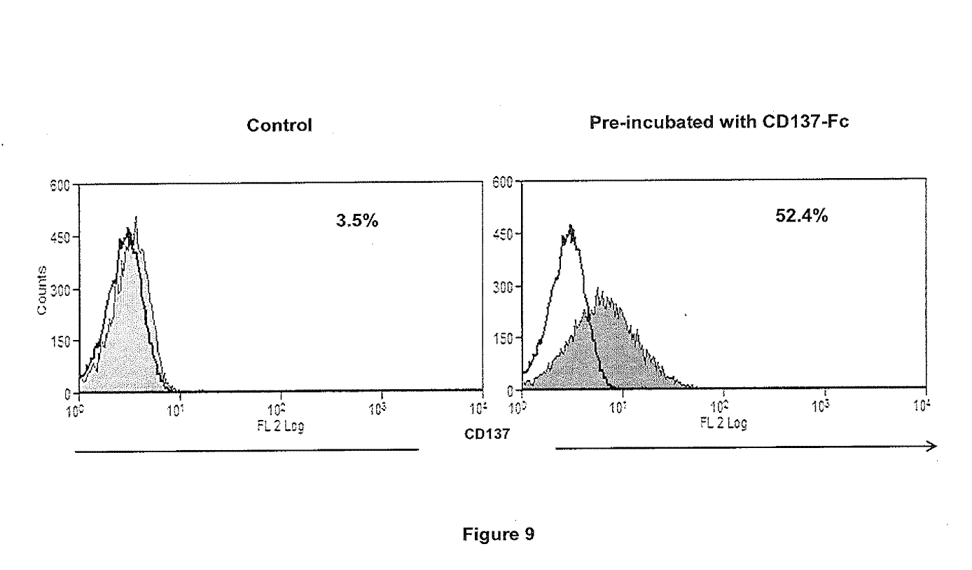

In several samples, very little or no CD137L could be detected by flow cytometry when anti-CD137L antibodies were used, but surface expression of CD137L could be observed when cells were pre-incubated with recombinant CD137 protein followed by subsequent detection using anti-CD137 antibodies (FIG. 9). Expression levels of CD137L did not associate with certain AML FAB subtypes, and were highly variable even between responsive samples. The CD137L expression did not appear to predict responsiveness to CD137 protein-induced differentiation (Table 4).

TABLE 4
AML samples tested

| | | | | CD137 | | | |
|---|---|---|---|---|---|---|---|
| Sample | | FAB | | ligand | Blasts | | Additional |
| No. | Age | subtype | Cell characteristics | (%) | (%) | Cytogenetics | Mutations |
| 1 | 43 | M1 | Blasts with | 45.9 | 88 | Normal | Mutations in |
| | | | minimal/no | | | | NPM1 and FLT-3 |
| | | | maturation | | | | |
| *2 | 78 | M1 | Blasts with | 72.5 | 89 | 47XY, +11 | n.a. |
| | | | minimal/no | | | | |
| | | | maturation | | | | |
| *3 | 57 | M1 | Blasts with | 39.9 | 88 | Normal | Mutations in |
| | | | minimal/no | | | | NPM1 and FLT-3 |
| | | | maturation | | | | |
| 4 | 58 | M2 | Blasts with | n.a. | 70 | 45XY, −7 | None detected |
| | | | minimal/no | | | | |
| | | | maturation | | | | |
| *5 | n.a. | M1/M2 | Blasts with | 30.6 | n.a. | n.a. | n.a. |
| | | | minimal/no | | | | |
| | | | maturation | | | | |
| 6 | 65 | n.a. | Blasts with | 14.5 | n.a. | n.a. | n.a. |
| | | | minimal/no | | | | |
| | | | maturation | | | | |
| 7 | 48 | n.a. | Blasts with | 0 | 88 | Normal | Mutation in cKit |
| | | | minimal/no | | | | (1621A > C) |
| | | | maturation | | | | |
| 8 | 67 | M2 | Blasts with | 91 | 37 | t(3; 4)(p23; q12) | None detected |
| | | | maturation | | | | |
| 9 | 65 | M2 | Blasts with | 0 | 45 | add(16)(q12) | Mutation in |
| | | | maturation | | | | CEBPA gene |
| *10 | 74 | M2 | Blasts with | 36.2 | 68 | Normal | n.a. |
| | | | maturation | | | | |
| *11 | 61 | M2 | Blasts with | 0 | n.a. | t(8; 21)(q22; q22)/ | None detected |
| | | | maturation | | | 45X, −X | |
| 12 | 60 | M2 | Blasts with | 19.4 | 51 | t(8; 21)(q22; q22)/ | None detected |
| | | | maturation | | | dup(7)(q22q32)/ | |
| | | | | | | 45X, −X | |
| 13 | 20 | M5a | Blasts with | 46.1 | 98 | t(11; 19)(q23; q13.3) | None detected |
| | | | maturation | | | | |
| 14 | 29 | n.a. | Blasts with | n.a. | 85 | Normal | n.a. |
| | | | maturation | | | | |
| 15 | n.a. | n.a. | Blasts with | n.a. | n.a. | n.a. | n.a. |
| | | | maturation | | | | |
| *16 | 56 | n.a. | Blasts with | 8.1 | 84 | Normal | Mutation in |
| | | | maturation | | | | FLT-3/ITD |
| 17 | 31 | M5b | Monocytic | 12.2 | 45 | t(11; 19)(q23; p13.1) | None detected |
| 18 | 70 | M5b | Monocytic | n.a. | 94 | Normal | n.a. |
| 19 | 45 | M6b | Monocytic | 10.9 | 82 | Normal | n.a. |
| 20 | n.a. | n.a. | Monocytic | n.a. | n.a. | n.a. | n.a. |
| 21 | 51 | M4 | Blasts with | 0 | 43 | Normal | Mutation in |
| | | | maturation | | | | CEBPA gene |

The samples are listed according to the FAB classification. The percentage of CD137L-expressing cells was determined by flow cytometry right after isolation of the cells. Samples marked by an asterisk (*) indicate AML cells that were resistant to CD137-induced differentiation. (n.a. means not available).

### Discussion

In this study, CD137L signaling was shown to induce changes in AML cell morphology, immunophenotype, cytokine release and cellular functions that are consistent with differentiation to more mature myeloid cells. These effects in AML cells mirror the differentiative effects of CD137L signaling in primary human monocytes (Ju, et al 2009, Kwajah and Schwarz 2010, Langstein, et al 1998, Lippert, et al 2008).

The therapeutic effects of CD137L signaling in AML may be twofold: (1) the increased differentiation should reduce the proliferative potential of the malignant cells, thereby slowing tumor progression (Stone, et al 2004); (2) CD137L signaling induces not just myeloid differentiation of AML cells but their differentiation to cells with DC-like properties (Kwajah and Schwarz 2010, Lippert, et al 2008) which can then present AML-associated antigens, and transform these former malignant AML cells to initiators of T cell responses against AML. Therefore, even if only a subset of AML cells responds with DC differentiation to the treatment with recombinant CD137 protein, it may still have a therapeutic effect on a much larger population of AML cells.

CD137 protein induced differentiation in 14 out of 20 (70%) of the primary AML samples tested and did so to varying degrees. Non-responsiveness of some of the AML samples to recombinant CD137-Fc protein could be due to poor viability of AML cells in vitro. The heterogeneity of the disease may also have contributed to the unresponsiveness of some AML samples, as the underlying cytogenetic abnormalities differ greatly even among responders, non-responders and samples of the same FAB subtype. It is also possible that some AML cells had acquired secondary mutations that nullify the effects of CD137L signaling. Nevertheless, patients responsive to a CD137 therapy could easily be identified by the in vitro assays employed in this study.

The signal transduction initiated by CD137L has only partly been studied and involves nuclear factor-κB (NF-κB), phosphatidylinositol-3-kinase/Akt and mitogen-activated protein kinase involving c-Jun-N-terminal kinase (JNK), p38 and extracellular signal-regulated kinase (Kim, et al 2011, Saito, et al 2004, Sollner, et al 2007). Except for a casein kinase recognition site, CD137L seems to lack conserved motifs for signal transduction in its short cytoplasmic domain (Watts, et al 1999). However, TNF receptor 1 was found to bind to CD137, and to be essential for CD137L signaling in the human monocytic cell line THP-1 (Moh, et al 2013).

Monocytic cells express CD137L naturally and constitutively (Shao and Schwarz 2011). For AML cells there may be even a selective advantage in expressing CD137L. CD137L on AML cells can crosslink CD137 on NK cells, and induce CD137 signaling which impairs the cytotoxic activity of NK cells and thereby facilitates escape of AML cells from immune surveillance (Baessler, et al 2010).

ATRA induces differentiation only in APL which is a major limitation of its therapeutic usefulness. In contrast, CD137 protein induced myeloid differentiation in cells of all FAB subtypes (M1, M2, M5a and M5b) tested. However, CD137L signaling does not support granulocyte differentiation (Jiang and Schwarz 2010). Therefore, it is unlikely that a differentiation therapy based on recombinant CD137 protein would be beneficial for APL.

CD137 protein needs to be immobilized in order to be able to crosslink CD137L. In vitro, that is achieved by coating CD137-Fc protein onto tissue culture plates. In vivo, recombinant CD137 protein could be immobilized onto micro- or nanoparticles. An alternative to using a recombinant CD137 protein could be an agonistic anti-CD137L antibody which would have the advantages that large scale production and regulatory approval are more streamlined for antibodies.

Potential side effects of a CD137-based differentiation therapy could include an induction of myelopoiesis (Tang, et al 2013). However, that may turn out to be beneficial as it may enhance the anti-tumor and general immunity in the patients.

CD137L has been demonstrated to be internalized upon the binding by CD137 (Ho, et al 2012) which may open the possibility of using recombinant CD137 protein as a carrier for cytotoxic drugs or radionucleotides into AML cells.

While the present study used recombinant CD137 protein to differentiate AML cells to DC, a related study found that AML-derived DC synergize with recombinant CD137L protein in stimulating T cell proliferation, expansion, their differentiation to effector cells (CD45RA⁺,CD27⁻) and IFN-γ release (Houtenbos, et al 2007). Therefore, the bidirectional signaling of the CD137 receptor/ligand system may be of twofold use in AML immunotherapy: CD137L signaling initiating DC differentiation, and CD137 signaling co-stimulating T cell activity.

Differential splicing and proteolytic cleavage can generate soluble forms of CD137 and CD137L, respectively (Setareh, et al 1995). Soluble CD137 antagonizes the activities of cell surface expressed CD137, while the activity of sCD137L is not known (Michel and Schwarz 2000). Interestingly, levels of sCD137L are prognostic of AML (Hentschel, et al 2006).

This study confirms that the myeloid differentiation induced by CD137L signaling observed in healthy monocytes and hematopoietic progenitor cells also applies to malignant cells in AML, and that treatment with a recombinant CD137 protein, which induces CD137L signaling, may be a new treatment option for AML.

### Example 2

For the in vitro studies described above and in Cheng et al., 2014 (Br J Haematol, 165(1):134-44), the recombinant CD137 protein was immobilized onto cell culture plates. However, there is a need to formulate the recombinant CD137 protein in such a way that it is (1) immobilized onto a carrier and (2) at the same time is soluble, so that it can circulate in the blood stream and reach the bone marrow. The aim in the present example was therefore to evaluate different methods of immobilization that are compatible with in vivo applications. Once optimized, these methods can be tested in murine models of acute myeloid leukemia (AML), which can be translated to AML treatment.

### 1. Formulation of Recombinant CD137 Protein for In Vivo Application

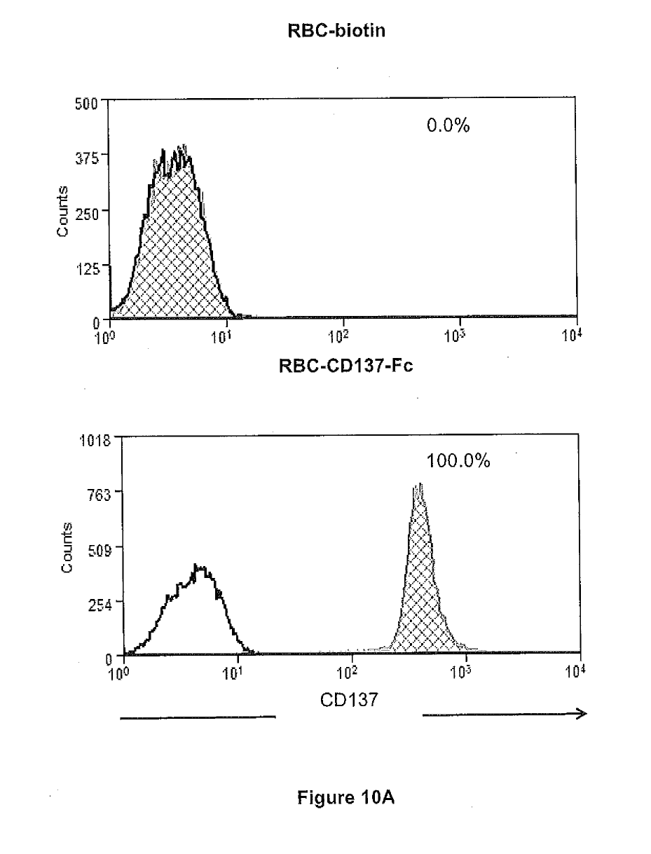

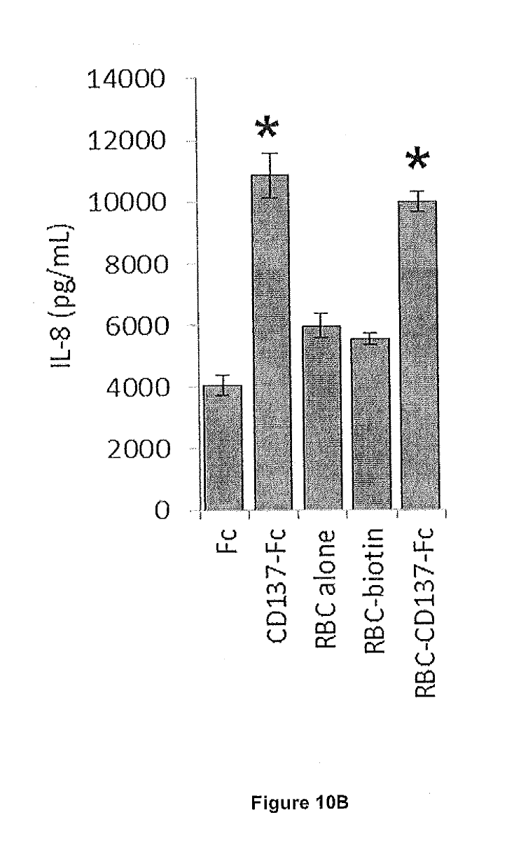

Two methods that have demonstrated some efficacy involved a cell-based approach. In the first approach, recombinant CD137 protein was conjugated to the surface membrane of red blood cells from healthy donors. This was achieved by biotinylation of both the recombinant CD137 protein and the red blood cell membrane proteins, which were subsequently linked by the addition of an avidin ‘bridge’. Freshly isolated red blood cells were biotinylated, bonded with avidin, then conjugated to biotinylated CD137-Fc. The presence of CD137-Fc on the red blood cells was determined by immunostaining followed by flow cytometry. High levels of CD137 were present on the surface membrane of the resulting red blood cells (FIG. 10A).

Monocytes were cultured for 5 days with RBCs conjugated with CD137-Fc or control RBCs conjugated only with biotin, or in dishes coated with immobilized CD137-Fc or Fc protein. The concentration of IL-8 in culture supernatants was determined by ELISA. Co-culture of these CD137-positive red blood cells with monocytes, which express CD137 ligand, induced an approximate 2-fold increase in IL-8 production compared to control cells (FIG. 10B).

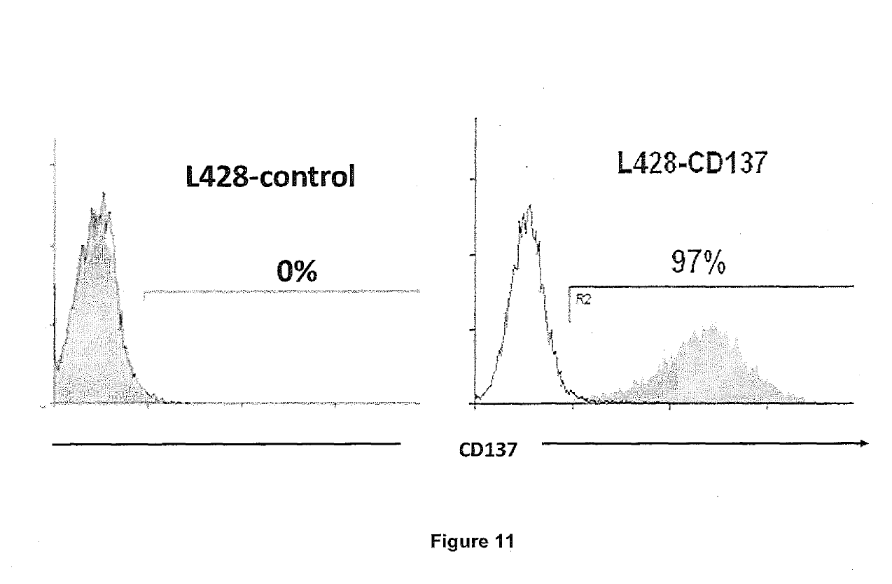

In the second approach, L428 cells that were transfected with an empty vector (L428-control) or with a CD137 expression vector (L428-CD137) were stained with an isotype or an anti-CD137 antibody. High levels of CD137 were ectopically expressed in a cell line that had been transfected with a lentiviral vector containing full-length CD137 cDNA (FIG. 11).

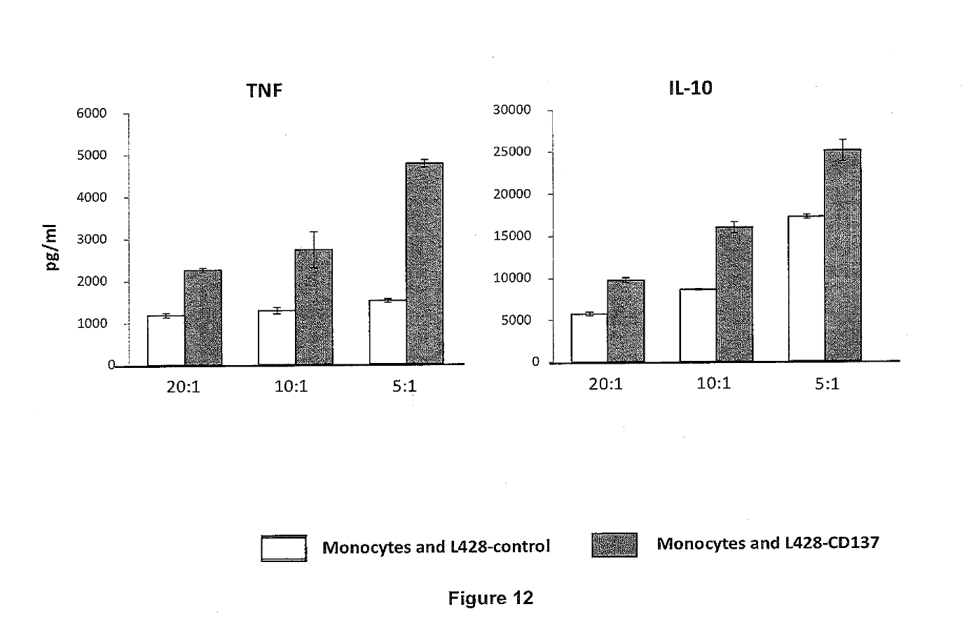

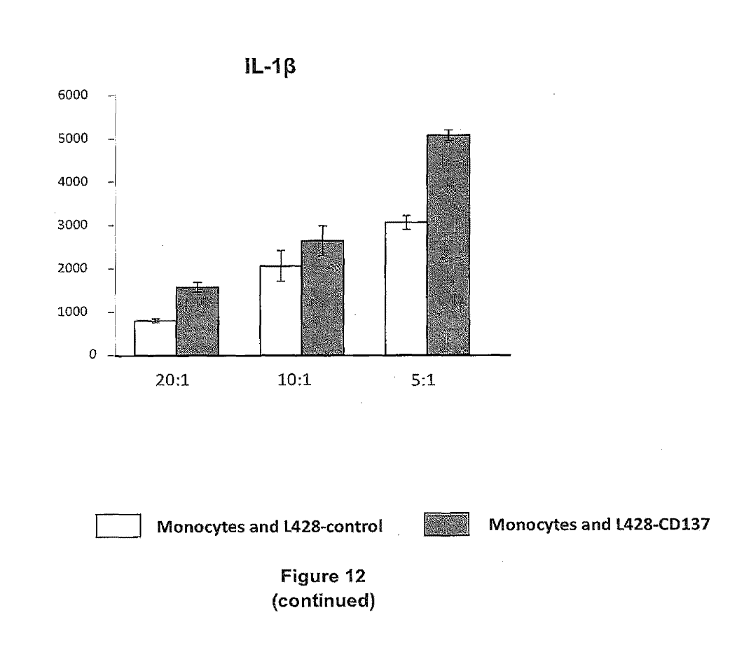

L428-control or L428-CD137 cells were co-cultured at ratios of 20:1, 10:1 or 5:1 with monocytes. After 24 h, the cytokine concentrations in co-culture supernatants were determined by ELISA. Co-culture of this CD137-expressing cell line (L428-CD137) with monocytes resulted in enhanced pro-inflammatory cytokine secretion compared to co-culture using the control, empty vector-transfected cells (L428-control) (FIG. 12). Both of these approaches prove that CD137, when expressed on the surface membrane of a cell, is able to cross-link CD137L on the ligand-bearing cell and to initiate CD137L signaling.

Another approach involves the exogenous and transient expression of CD137 on the surface of T cells. The mRNA of a truncated form of CD137 is generated by in vitro transcription from a plasmid vector, pvaxl, containing the sequence for the extracellular and transmembrane domain (but not the cytoplasmic tail) of CD137. This mRNA is then introduced into primary, unstimulated T cells via electroporation, after which the truncated CD137 protein is expressed on the T cell surface. After confirming that these CD137-positive T cells are able to induce CD137L-signaling in ligand-bearing cells in vitro, the in vivo effects will be examined using murine models of leukemia.

### 2. In Vivo Proof of Concept Studies in Mice

Two murine models for AML have been established, one with a mutation in the gene encoding Core-binding factor 3 subunit (Cbfβ) which is frequently mutated in human AML patients, and another model in which human AML cells are transplanted into immunodeficient mice. It was demonstrated that bone marrow cells derived from conditional Cbfβ knock-out mice are indeed responsive to CD137-induced differentiation in vitro. Loss of Cbfβ in these mice led to a differentiation block in the myeloid lineage and an expansion of the haematopoietic stem cell compartment which reflects the pathogenesis of AML.

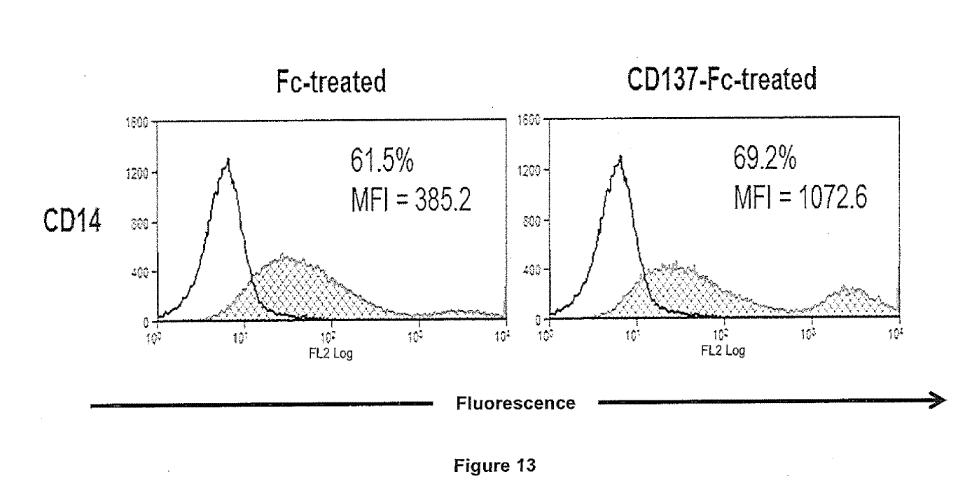

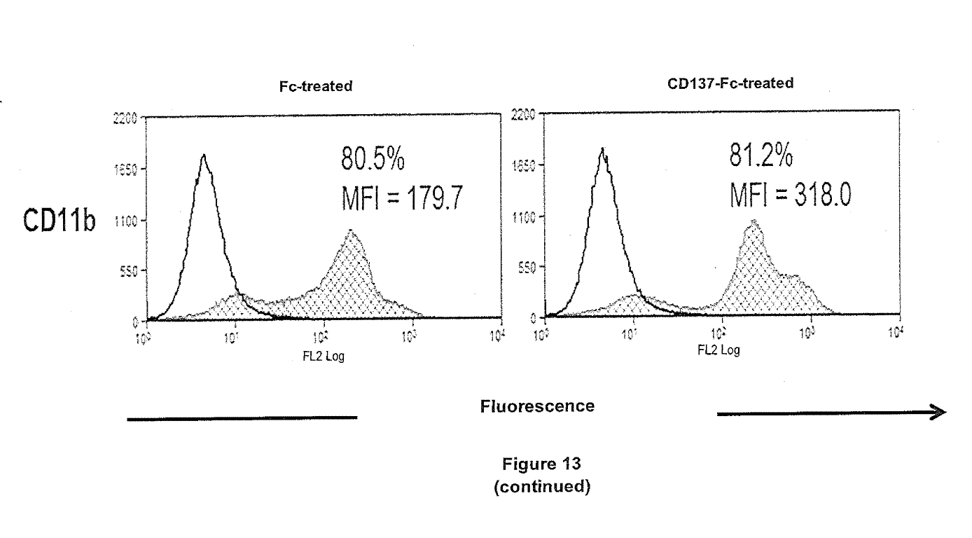

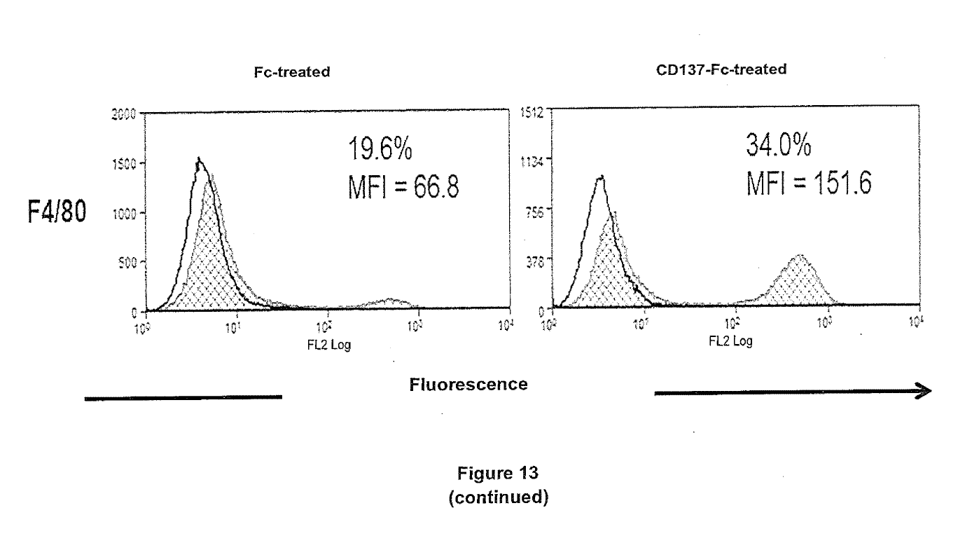

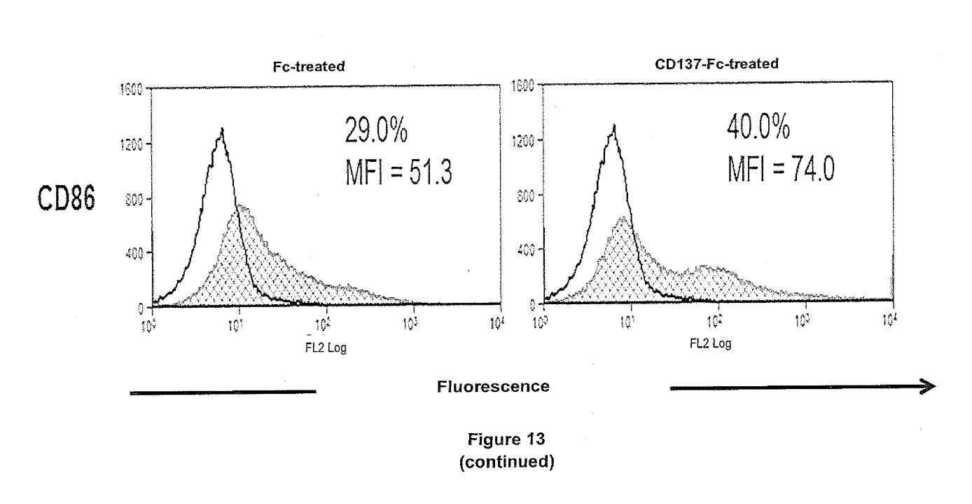

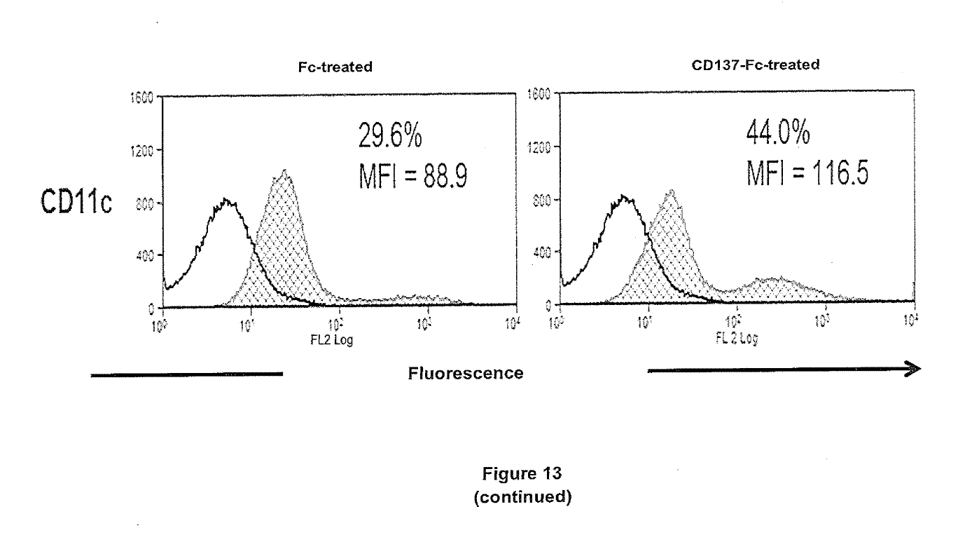

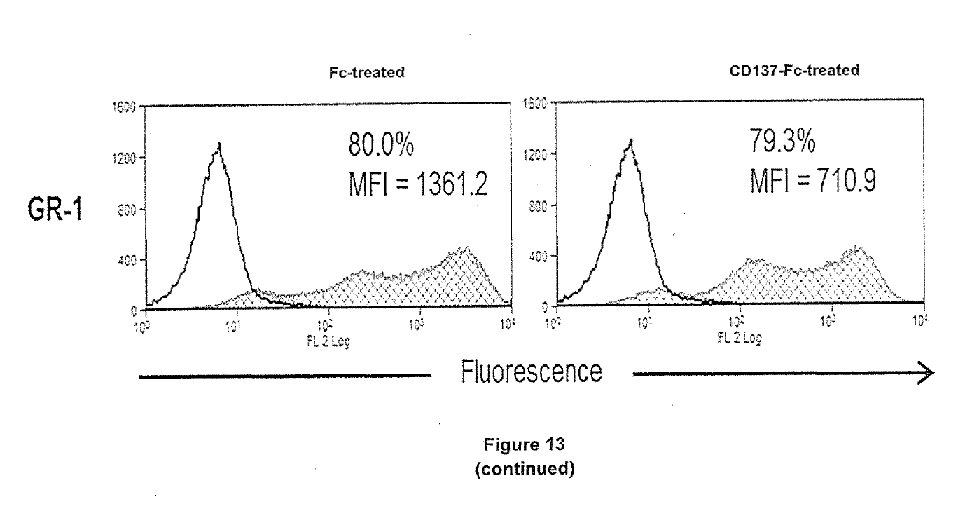

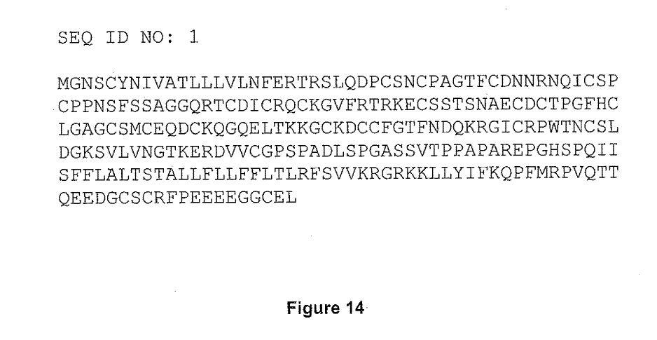

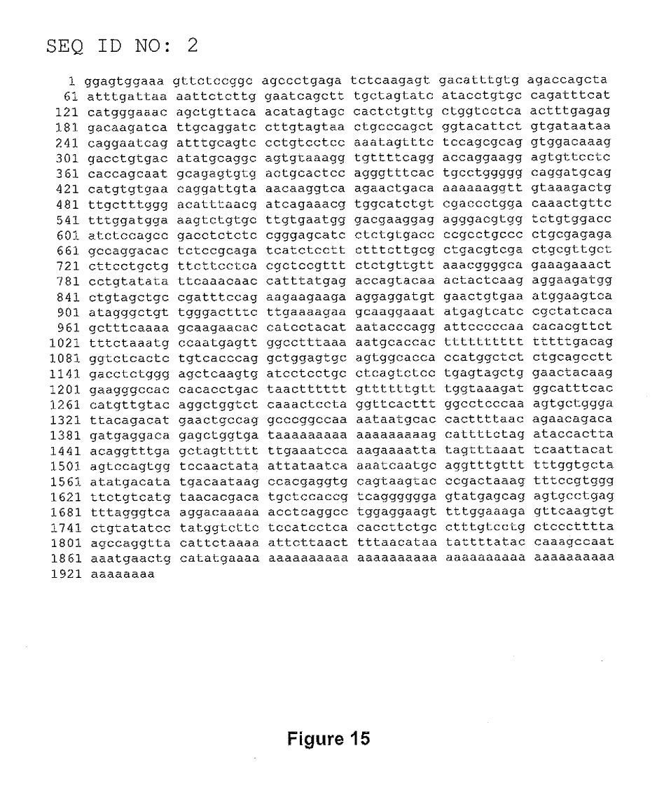

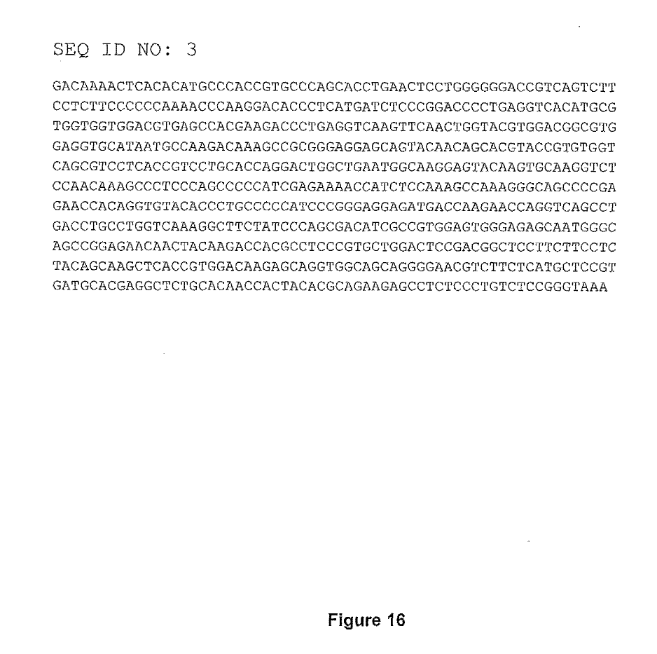

Bone marrow cells from Cbfβ knockout mice were cultured for 5 days in dishes coated with immobilized Fc or CD137-Fc protein. Cells were then harvested and expression of surface markers was determined by immunostaining followed by flow cytometry. Treatment of the bone marrow cells with recombinant CD137 protein immobilized onto a tissue culture dish induced changes in the myeloid cell immunophenotype that were consistent with differentiation/maturation (FIG. 13). An induction of the differentiation markers CD14, CD11b, CD11c, CD86 and F4/80 was observed.

### Applications

The agonists described herein may be used for the treatment of cancer, particularly myeloid malignancies, such as AML. The agonists may also be used to induce myeloid differentiation in AML cells and prevent cancer progression. They may also be used to activate immune cells to boost immune response to cancer.

Furthermore, the agonists may be used as a tool to determine whether a patient is likely to respond to therapy with a CD137L agonist. For example, the agonists may be coupled to a detectable agent and be applied to cells or tissue samples in order to measure levels of CD137L expression on a cancer cell (such as AML cells) from a cancer patient, which may then be used to determine whether the patient is likely to respond to therapy with a CD137L agonist.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

### REFERENCES

Baessler, T., Charton, J. E., Schmiedel, B. J., Grunebach, F., Krusch, M., Wacker, A., Rammensee, H. G. & Salih, H. R. (2010) CD137 ligand mediates opposite effects in human and mouse NK cells and impairs NK-cell reactivity against human acute myeloid leukemia cells. Blood, 115, 3058-3069.
Cheng, K., Wong, S. C., Linn, Y. C., Ho, L. P., Chng, W. J. & Schwarz, H. (2014) CD137 ligand signalling induces differentiation of primary acute myeloid leukaemia cells. Br J Haematol, 165, 134-144.
Drenkard, D., Becke, F. M., Langstein, J., Spruss, T., Kunz-Schughart, L. A., Tan, T. E., Lim, Y. C. and Schwarz, H. (2007). CD137 is expressed on bood vessel walls at sites of inflammation and enhances monocyte migratory activity. FASEB Journal., 21, 456-463.
Eissner, G., Kolch, W. & Scheurich, P. (2004) Ligands working as receptors: reverse signaling by members of the TNF superfamily enhance the plasticity of the immune system. Cytokine Growth Factor Review, 15, 353-366.
Hentschel, N., Krusch, M., Kiener, P. A., Kolb, H. J., Salih, H. R. & Schmetzer, H. M. (2006) Serum levels of sCD137 (4-1BB) ligand are prognostic factors for progression in acute myeloid leukemia but not in non-Hodgkin's lymphoma. European Journal of Haematology, 77, 91-101.
Ho, W. T., Pang, W. L., Chong, S. M., Castello, A., Al-Salam, S., Tan, T. E., Moh, M. C., Koh, L. K., Gan, S. U., Cheng, C. K. & Schwarz, H. (2012) Expression of CD137 on Hodgkin and Reed-Sternberg Cells Inhibits T cell Activation by Eliminating CD137 Ligand Expression. Cancer Research, 73, 652-661.
Houtenbos, I., Westers, T. M., Dijkhuis, A., de Gruijl, T. D., Ossenkoppele, G. J. & van de Loosdrecht, A. A. (2007) Leukemia-specific T-cell reactivity induced by leukemic dendritic cells is augmented by 4-1BB targeting. Clinical Cancer Research, 13, 307-315.
Jiang, D., Chen, Y. & Schwarz, H. (2008a) CD137 induces proliferation of murine hematopoietic progenitor cells and differentiation to macrophages. Journal of Immunology, 181, 3923-3932.
Jiang, D. & Schwarz, H. (2010) Regulation of Granulocyte and Macrophage Populations of Murine Bone Marrow Cells by G-CSF and CD137 Protein. PLoS One, 5, e15565.
Jiang, D., Yue, P. S., Drenkard, D. & Schwarz, H. (2008b) Induction of proliferation and monocytic differentiation of human CD34+cells by CD137 ligand signaling. Stem Cells, 26, 2372-2381.
Ju, S., Ge, Y., Qiu, H., Lu, B., Qiu, Y., Fu, J., Liu, G., Wang, Q., Hu, Y., Shu, Y. & Zhang, X. (2009) A novel approach to induce human DCs from monocytes by triggering 4-1BBL reverse signaling. International Immunology, 21, 1135-1144.
Kim, J. D., Lee, E. A., Quang, N. N., Cho, H. R. & Kwon, B. (2011) Recombinant TAT-CD137 Ligand Cytoplasmic Domain Fusion Protein Induces the Production of IL-6 and TNF-alpha in Peritoneal Macrophages. Immune Network, 11, 216-222.
Kim, Y. J., Li, G. & Broxmeyer, H. E. (2002) 4-1BB ligand stimulation enhances myeloid dendritic cell maturation from human umbilical cord blood CD34+ progenitor cells. Journal of Hematotherapy and Stem Cell Research, 11, 895-903.
Koeffler, H. P. (2010) Is there a role for differentiating therapy in non-APL AML? Best Practice & Research Clinical Haematology, 23, 503-508.
Kwajah, M. M. S. & Schwarz, H. (2010) CD137 ligand signaling induces human monocyte to dendritic cell differentiation. European Journal of Immunology, 40, 1938-1949.
Laderach, D., Wesa, A. & Galy, A. (2003) 4-1BB-ligand is regulated on human dendritic cells and induces the production of IL-12. Cellular Immunology, 226, 37-44.
Langstein, J., Becke, F. M., Sollner, L., Krause, G., Brockhoff, G., Kreutz, M., Andreesen, R. & Schwarz, H. (2000) Comparative analysis of CD137 and LPS effects on monocyte activation, survival, and proliferation. Biochemical and Biophysical Research Communications, 273, 117-122.
Langstein, J., Michel, J., Fritsche, J., Kreutz, M., Andreesen, R. & Schwarz, H. (1998) CD137 (ILA/4-1BB), a member of the TNF receptor family, induces monocyte activation via bidirectional signaling. Journal of Immunology, 160, 2488-2494.
Langstein, J., Michel, J. & Schwarz, H. (1999) CD137 induces proliferation and endomitosis in monocytes. Blood, 94, 3161-3168.
Langstein, J. & Schwarz, H. (1999) Identification of CD137 as a potent monocyte survival factor. Journal of Leukocyte Biology, 65, 829-833.
Lee, S. W. & Croft, M. (2009) 4-1BB as a therapeutic target for human disease. Advances in Experimental Medicine and Biology, 647, 120-129.
Lippert, U., Zachmann, K., Ferrari, D. M., Schwarz, H., Brunner, E., Latif, A. H., Neumann, C. & Soruri, A. (2008) CD137 ligand reverse signaling has multiple functions in human dendritic cells during an adaptive immune response. European Journal of Immunology, 38, 1024-1032.
Michel, J. & Schwarz, H. (2000) Expression of soluble CD137 correlates with activation-induced cell death of lymphocytes. Cytokine, 12, 742-746.
Moh, M. C., Lorenzini, P. A., Gullo, C. & Schwarz, H. (2013) Tumor necrosis factor receptor 1 associates with CD137 ligand and mediates its reverse signaling. FASEB Journal, 27, 2957-2966.
Petrie, K., Zelent, A, & Waxman, S. (2009) Differentiation therapy of acute myeloid leukemia: past, present and future. Current Opinion in Hematology, 16, 84-91.
Saito, K., Ohara, N., Hotokezaka, H., Fukumoto, S., Yuasa, K., Naito, M., Fujiwara, T. & Nakayama, K. (2004) Infection-induced up-regulation of the costimulatory molecule 4-1BB in osteoblastic cells and its inhibitory effect on M-CSF/RANKL-induced in vitro osteoclastogenesis. Journal of Biological Chemistry, 279, 13555-13563.
Schwarz, H. (2005) Biological activities of reverse signal transduction through CD137 ligand. Journal of Leukocyte Biology, 77, 281-286.
Schwarz, H., Blanco, F. J., von, K. J., Valbracht, J. & Lotz, M. (1996) ILA, a member of the human nerve growth factor/tumor necrosis factor receptor family, regulates T-lymphocyte proliferation and survival. Blood, 87, 2839-2845.
Setareh, M., Schwarz, H. & Lotz, M. (1995) A mRNA variant encoding a soluble form of 4-1BB, a member of the murine NGF/TNF receptor family. Gene, 164, 311-315.
Shao, Z. & Schwarz, H. (2011) CD137 ligand, a member of the tumor necrosis factor family, regulates immune responses via reverse signal transduction. Journal of Leukocyte Biology, 89, 21-29.
Sollner, L., Shaqireen, D. O. K., Wu, J. T. & Schwarz, H. (2007) Signal transduction mechanisms of CD137 ligand in human monocytes. Cellular Signalling, 19, 1899-1908.
Stone, R. M., O'Donnell, M. R. & Sekeres, M. A. (2004) Acute myeloid leukemia. Hematology Am Soc Hematol Educ Program, 98-117.
Tamada, K. & Chen, L. (2006) Renewed interest in cancer immunotherapy with the tumor necrosis factor superfamily molecules. Cancer Immunology and Immunotherapy, 55, 355-362.
Tang, Q., Jiang, D., Alonso, S., Pant, A., Gomez, J. M., Kemeny, D. M., Chen, L. & Schwarz, H. (2013) CD137 ligand signaling enhances myelopoiesis during infections. European Journal of Immunology, 43, 1555-1567.
Thum, E., Shao, Z. & Schwarz, H. (2009) CD137, implications in immunity and potential for therapy. Frontiers in Bioscience, 14, 4173-4188.
Wang, C., Lin, G. H., McPherson, A. J. & Watts, T. H. (2009) Immune regulation by 4-1BB and 4-1BBL: complexities and challenges. Immunolgical Reviews, 229, 192-215.
Watts, A. D., Hunt, N. H., Wanigasekara, Y., Bloomfield, G., Wallach, D., Roufogalis, B. D. & Chaudhri, G. (1999) A casein kinase I motif present in the cytoplasmic domain of members of the tumour necrosis factor ligand family is implicated in ‘reverse signalling’. EMBO Journal, 18, 2119-2126.

1. A method of treating cancer comprising administering an agonist of CD137 ligand (CD137L) to a subject in need thereof.

2. The method according to claim 1, wherein the cancer is selected from the group consisting of a myeloid malignancy, a lymphoid malignancy, and multiple myeloma.

3. The method according to claim 2, wherein the myeloid malignancy is selected from the group consisting of acute myeloid leukemia (AML), chronic myeloid leukemia (CML), acute promyelocytic leukemia (APL), chronic myelomonocytic leukemia (CMML), myelodysplastic syndrome, and juvenile myelomonocytic leukemia.

4. The method according to claim 2, wherein the lymphoid malignancy is selected from the group consisting of chronic lymphocytic leukemia (CLL), acute lymphoblastic leukemia (ALL), B cell lymphoma, and mantle cell lymphoma (MCL).

5. The method according to claim 3, wherein the AML comprises a FAB subtype selected from the group consisting of M0, M1, M2, M3, M4, M5, M5a, M5b, M6 and M7.

6. The method according to claim 1, wherein the agonist of CD137L is selected from the group consisting of a fragment of CD137 protein, a recombinant CD137 protein, a CD137 fusion protein, an anti-CD137L antibody or a fragment thereof, a peptide, a small molecule, an aptamer, and an antibody mimetic.

7. The method according to claim 6, wherein the recombinant CD137 protein comprises the extracellular domain of CD137 fused to the Fc region of human IgG (CD137-Fc).

8. The method according to claim 6, wherein the anti-CD137L antibody or fragment thereof comprises an agonistic anti-CD137L antibody or a fragment thereof.

9. The method according to claim 1, wherein the agonist of CD137L is immobilized on a particle.

10. The method according to claim 1, wherein the agonist of CD137L is coupled to an agent selected from the group consisting of cytotoxic drugs, radiotherapeutics, chemotherapeutics, enzyme, and a carrier molecule.

11. The method according to claim 1, wherein the cancer is not APL or AML subtype M3.

12-33. (canceled)

34. A method for inducing differentiation in cancer cells, comprising administering an agonist of CD137 ligand (CD137L) to said cancer cells, wherein the agonist of CD137L is selected from the group consisting of a fragment of CD137 protein, a recombinant CD137 protein, a CD137 fusion protein, an anti-CD137L antibody or a fragment thereof, a peptide, a small molecule, an aptamer, and an antibody mimetic.

35. A method for delivering a therapeutic or diagnostic agent to a cell in need thereof, comprising coupling an agonist of CD137 ligand (CD137L) to an agent selected from the group consisting of cytotoxic drugs, radiotherapeutics, chemotherapeutics, enzyme, and a carrier molecule, wherein the agonist of CD137L is selected from the group consisting of a fragment of CD137 protein, a recombinant CD137 protein, a CD137 fusion protein, an anti-CD137L antibody or a fragment thereof, a peptide, a small molecule, an aptamer, and an antibody mimetic.


SEQUENCE LISTING (3 sequences)

SEQ ID NO: 1 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 2 - DNA, 1928 bp, Homo sapiens
ggagtggaaagttctccggcagccctgagatctcaagagtgacatttgtgagaccagctaatttgattaaaattctcttggaatcagctttgctagtatcatacctgtgccagatttcatcatgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtgaaatggaagtcaatagggctgttgggactttcttgaaaagaagcaaggaaatatgagtcatccgctatcacagctttcaaaagcaagaacaccatcctacataatacccaggattcccccaacacacgttcttttctaaatgccaatgagttggcctttaaaaatgcaccactttttttttttttttgacagggtctcactctgtcacccaggctggagtgcagtggcaccaccatggctctctgcagccttgacctctgggagctcaagtgatcctcctgcctcagtctcctgagtagctggaactacaaggaagggccaccacacctgactaacttttttgttttttgtttggtaaagatggcatttcaccatgttgtacaggctggtctcaaactcctaggttcactttggcctcccaaagtgctgggattacagacatgaactgccaggcccggccaaaataatgcaccacttttaacagaacagacagatgaggacagagctggtgataaaaaaaaaaaaaaaaaagcattttctagataccacttaacaggtttgagctagtttttttgaaatccaaagaaaattatagtttaaattcaattacatagtccagtggtccaactataattataatcaaaatcaatgcaggtttgttttttggtgctaatatgacatatgacaataagccacgaggtgcagtaagtacccgactaaagtttccgtgggttctgtcatgtaacacgacatgctccaccgtcaggggggagtatgagcagagtgcctgagtttagggtcaaggacaaaaaacctcaggcctggaggaagttttggaaagagttcaagtgtctgtatatcctatggtcttctccatcctcacaccttctgcctttgtcctgctcccttttaagccaggttacattctaaaaattcttaacttttaacataatattttataccaaagccaataaatgaactgcatatgaaaaaaaaaaaaaaaaaaaaaaaaaaaaaaaaaaaaaaaaaaaaaaaaaaaa

SEQ ID NO: 3 - DNA, 681 bp, artificial sequence
  hIgG1-Fc region
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa